(19) United States
(12) Patent Application Publication
(10) Pub. No.: US 2024/0304282 A1
(43) Pub. Date: Sep. 12, 2024

(54) OPTIMIZED EXPRESSION IN TARGET ORGANISMS

(71) Applicant: RAMOT AT TEL-AVIV UNIVERSITY LTD., Tel Aviv (IL)

(72) Inventor: Tamir TULLER, Tel Aviv (IL)

(21) Appl. No.: 18/585,138

(22) Filed: Feb. 23, 2024

Related U.S. Application Data
(63) Continuation of application No. PCT/IL2022/050930, filed on Aug. 25, 2022.
(60) Provisional application No. 63/236,814, filed on Aug. 25, 2021.

(51) Int. Cl. G16B 30/20

(52) U.S. Cl. CPC G16B 30/20

(57) ABSTRACT

Computerized methods for engineering a nucleic acid molecule comprising a coding region optimized for expression in a first set of organisms and deoptimized for expression in a second set of organisms are provided.

### CROSS REFERENCE TO RELATED APPLICATIONS

This application is a Bypass Continuation of PCT Patent Application No. PCT/IL2022/050930 having International filing date of Aug. 25, 2022, which claims the benefit of priority of U.S. Provisional Patent Application No. 63/236,814, filed Aug. 25, 2021, the contents of which are all incorporated herein by reference in their entirety.

### FIELD OF INVENTION

The present invention is in the field of protein expression optimization.

### BACKGROUND OF THE INVENTION

The term “microbiome” is defined as the community of different microorganisms that coexist in an environment. Nearly every system, from natural to synthetic, is populated by a unique and diverse community of organisms, which continuously interact among themselves and with their environment. Early studies of the field have shown that the animal's microbiome has a noticeable effect on key features including their host's fitness and lifespan. Research regarding the human and animal microbiome in the past years has led to truly impactful results that provide new understanding of the mechanisms of host-microbiome interactions and their key influence of various physiological and even psychological factors. Research has established the tendency of microbiome composition to respond and further modulate environmental changes, marking them as a desirable target for bioengineering, promoting the development of diverse engineering methodologies.

Some techniques such as microbiome directed evolution, genomic engineering, and others that require transplant of new or otherwise altered bacteria into the environment, have succeeded in some conditions usually for shorter time frames. The main reason for that is that the transplanted bacteria are less adapted to the new environment and therefore are disadvantaged compared to the native bacteria in the competition over the environmental niche. Other methods aim to engineer the bacterial microbes that are already present in the environment and have mainly focused on the transformation vector itself. Most of these systems have been developed specifically for a certain environment and cannot be easily applied in others, however the more crucial problem arises once the new genetic information is introduced to the environment. Bacteria constantly share genetic information through various methods of horizontal gene transfer, and in most cases, in order for the engineering process to be effective and accurate, it should be introduced precisely to the wanted hosts. Moreover, due to the innate variations and unpredictability of biological systems, introduction of new genetic information to an uncontrolled environment may cause unprecedented ecological impacts. A method of recoding genetic information in order to modulate gene expression in various organisms while simultaneously blocking expression in other organisms is therefore greatly needed.

### SUMMARY OF THE INVENTION

The present invention provides computerized methods for engineering a nucleic acid molecule comprising a coding region optimized for expression in a first set of organisms and deoptimized for expression in a second set of organisms.

According to a first aspect, there is provided a computerized method for engineering a nucleic acid molecule comprising a coding region optimized for expression of the coding region in a first set of organisms and deoptimized for expression of the coding region in a second set of organisms,

the method comprising at least one of:
a. calculating a codon usage bias (CUB) of the first set of organisms, and a CUB of the second set of organisms and replacing at least one codon of a nucleotide sequence of the coding region with a synonymous codon, wherein the synonymous codon is selected for in the first set of organisms based on the calculated CUB and deselected for in the second set of organisms based on the calculated CUB;
b. receiving a first list of sequences of regulatory elements of highly expressed genes in the first set of organisms and a second list sequences of regulatory elements of highly expressed genes in the second set of organisms, selecting sequence motifs enriched in the first list and depleted in the second list, engineering an artificial regulatory element comprising a plurality of the selected sequence motifs and operably linking the artificial regulatory element to the coding region in the nucleic acid molecule;
c. determining target sequences of DNA cleaving agents expressed only by the first set of organisms and target sequences of DNA cleaving agents expressed only by the second set of organisms and altering a sequence of the nucleic acid molecule to include at least one of the target sequences of DNA cleaving agents expressed only by the second set of organisms or to remove at least one target sequence of DNA cleaving agents expressed only by the first set of organisms;
d. extracting sequence features that promote replication from origins of replication (ORI) from the first set of organisms and the second set of organisms, generating an artificial ORI in the nucleic acid molecule that is enriched for sequence features from the first set of organisms and depleted of sequence features from the second set of organism;
e. identifying at least one gene highly expressed in the second set of genes that is not highly expressed in the first set of genes and introducing into an open reading frame of the nucleic acid molecule at least a portion of the at least one gene highly expressed in the second set of genes; and
f. optimizing intergenic sequence in the nucleic acid molecule by enriching the intergenic sequence with uptake signal sequences (USS) from the first set of organisms and depleting the intergenic sequence of USS from the second set of organisms;

thereby engineering a nucleic acid molecule.

According to some embodiments, the CUB is calculated by a tRNA adaptation index (tAI), by a codon adaptation index (CAI) or by typical decoding rate (TDR).

According to some embodiments, all codons of the nucleotide sequence that can be, are replaced with a synonymous codon selected for in the first set of organisms based on the CUB and deselected for in the second set of organisms based on the CUB.

According to some embodiments, the regulatory elements are promoters.

According to some embodiments, the highly expressed genes are selected based on a predetermined threshold of a percentage of all genes.

According to some embodiments, the highly expressed genes are inferred based on CUB rankings of coding sequences of all genes in each organism.

According to some embodiments, selecting sequence motifs comprises employing a hidden Markov model.

According to some embodiments, engineering an artificial regulatory element comprises selecting an endogenous regulatory element from the first list which is highly enriched for the selected sequence motifs.

According to some embodiments, selecting an endogenous regulatory element comprises ranking the regulatory elements from the first list based on their enrichment with the selected sequencing motifs and the significance of enrichment of the selected sequencing motifs in the first list.

According to some embodiments, the ranking comprises using a k−1 order Markov model.

According to some embodiments, the computerized method further comprises producing at least one mutation in the endogenous regulatory element that produces at least one selected sequence motif.

According to some embodiments, the altering a sequence occurs with the coding region, or within a regulatory region that is required for or enhances expression of the coding region.

According to some embodiments, the altering is with the coding region and does not alter an amino acid sequence encoded by the coding sequence.

According to some embodiments, the DNA cleaving agent is a DNA cleaving protein.

According to some embodiments, the DNA cleaving agent is selected from a restriction enzyme and a genome editing protein.

According to some embodiments, the genome editing protein is a clustered regulatory interspaced short palindromic repeats (CRISPR) protein.

According to some embodiments, the altering a sequence comprises producing a PAM sequence of a CRISPR protein and a spacer sequence expressed only by the second set of organisms.

According to some embodiments, the DNA cleaving agent is a restriction enzyme and the altering a sequence comprises producing at least one palindromic target sequences of a restriction enzyme expressed only by the second set of organisms or mutating a palindromic target sequence of a restriction enzyme expressed only by the first set of organisms.

According to some embodiments, generating an artificial ORI comprises performing hierarchical clustering of the extracted sequence features that promote replication from ORI from the first list of organisms and if a distance between clusters is greater than a predetermined threshold including all clusters in the nucleic acid molecule and if the distance is less than the predetermined threshold generating a single cluster related to all ORI sequences in all the clusters.

According to some embodiments, the computerized method comprises producing at least one mutation in the artificial ORI that produces a sequence feature from the first set of organisms or that removes a sequence feature from the second set of organisms.

According to some embodiments, the computerized method comprises selecting at least one feature from at least one clusters from the first set of organisms and removing at least one feature from at least one cluster from the second set of organisms.

According to some embodiments, the at least one gene highly expressed in the second set of organisms is an essential gene.

T According to some embodiments, the portion of the at least one gene highly expressed is the second set of organisms acts as an siRNA against the at least one highly expressed gene.

According to some embodiments, the nucleic acid molecule is a DNA molecule.

According to some embodiments, the nucleic acid molecule is a plasmid.

According to some embodiments, the first set of organisms, the second set of organisms or both are bacteria.

According to some embodiments, the computerized method further comprises outputting an artificial sequence of the engineered nucleic acid molecule.

According to another aspect, there is provided an engineered nucleic acid molecule produced by a computerized method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Illustration of the main genetic components of a gene transfer plasmid that can be optimized as part of an embodiment of the invention to modulate expression of the designed plasmid only in some of the organisms of a target microbiome.

FIG. 2: A schematic of a method of the invention for translation optimization.

FIG. 3: One embodiment of the translation (CUB) optimization algorithm of the invention. One hill climbing iteration of the translation optimization algorithm is shown. The first step is to define the wanted and un-wanted hosts (1). The second step is to calculate the CUB score of each organism for all codons of amino acid A to Ai (score_Ai) and then calculate the mean (μ_CUBi) and the standard deviation (σ_CUBi) of the CUB scores (2). Finally, an optimization score is calculated for each synonymous codon. All the amino acid codons in the initial sequence are switched to the codon with the maximal optimization score as calculated (3).

FIG. 4: A line graphs of scores for E. coli and B. subtilis optimization and deoptimization by CAI, tAI and TDR.

FIG. 5: A schematic of a method of the invention for transcriptional optimization.

FIG. 6: One embodiment of the promoter (transcription) optimization algorithm of the invention. Promoter and intergenic regions sequences are extracted for every wanted and unwanted host (1) and are used as inputs for STREME software tool to find transcription enhancing motifs for wanted hosts and transcription anti-motifs for unwanted hosts (2). Transcription enhancing motifs with high correlation to other transcription enhancing motifs and/or other anti-motifs which have the highest coverage for the given microbiome population are chosen for the final motif set (3). Motifs in the final motif set are used to score potential candidate promoters using the MAST software tool (4). Synthetic promoter versions are created for top ranked promoters to further tailor the sequences based on alignment to the discovered transcription promoting motifs (5).

FIG. 7: Dot plots of scores for promoter sequences from (top) B. subtilis and (bottom) E. coli based on motifs found in the two organisms.

FIG. 8: A schematic of a method of the invention for restriction enzyme site optimization. The restriction enzymes (triangles) are extracted from the optimized and deoptimized organisms respectively (1). The selected restriction sites (squares) are the sites that contain the restriction enzymes exclusive to the deoptimized organisms (2). Then, the restriction sites from the deoptimized organisms are added to the sequence and the restriction sites from the optimized organisms are removed to yield the final product (3).

FIG. 9: One embodiment of the restriction site algorithm of the invention. The restriction enzymes (triangles) are extracted from the wanted and unwanted hosts respectively; the recognition sites of the enzymes are illustrated by squares.

FIG. 10: A schematic of a method of the invention for CRISPR site optimization.

FIG. 11: A schematic of a method of the invention for ORI optimization.

FIGS. 12A-C: Heatmaps showing results from a single run of the translation (CUB) optimization algorithm. Translation efficiency optimization of (12A) the A1, A2. thaliana microbiome, using the calculated CUB scores of all codons, (12B) the initial scores of the ZorA gene, and (12C) the final scores of the gene. The upper half of the organisms (1-16) were defined as the optimized organisms and the lower half as the deoptimized organisms (17-34).

FIGS. 13A-B: Final test of algorithm resolution and scale up. (13A) Bar graph showing dependence of the algorithm on microbiome size. (10 different random splits of chosen sizes, averaged). (13B) Dot plot showing the correlation between the performance of the model and the evolutionary distance between a pair of species (defined as the number of differences in the alignments of the 16S rRNA sequences).

FIG. 14: Bar charts of E-value scores from a MAST run for a final motif set constructed for a pair of species from the Arthrobacter family, including the wanted host Arthrobacter pascens (left) and unwanted host Arthrobacter tumbea (right). In both the mean and median E-values are indicated. Wanted host motifs were calculated by a STREME run using promoter sequences as primary set and intergenic regions as control set. Unwanted host anti-motifs were calculated by a STREME run using intergenic regions as primary set and promoter sequences as control set. Mean and median E-values of the wanted host are lower than mean and median E-values for the unwanted host, with a p-value of 7.184e-8.

FIGS. 15A-B: (15A) Bar graph of E-value scores from a MAST run for a final motif set constructed for randomized MGnify sub-microbiomes of different sizes. The count of wanted and unwanted hosts was set to half the size of the microbiome. Only values from the 5th-percentile of the E-values calculated for the promoters of each host were considered. E-values for each group (wanted/unwanted) were calculated as the median of the median of the values of each host in the group. Test was repeated 10 times for each microbiome size. (15B) Meta analysis of MGnify microbiomes.

FIGS. 16A-D: Characteristics of the engineered sequence. Random samples of 10 to 50 species were selected, and randomly split into 2 subgroups—of wanted organisms, and one of unwanted organisms. After applying the model to the defined microbiome, line graphs showing (16A) the number of sites incorporated in the final sequence from each one of the two groups, (16B) the number of organisms that have a corresponding site, and (16C) the percent of organisms that have a corresponding site were generated. (16D) Line graph of the normalized presence of restriction sites recognized by the wanted and unwanted hosts. An average of 10 runs in each condition are shown.

FIGS. 17A-D: ORF modification alters the growth of deoptimized bacteria. (17A) Representative growth curves for B. subtilis (top) and in E. coli (bottom). Control (black dashed curve) stands for bacteria containing the same plasmid backbone that lacks the mCher-ry gene. mCherry (red dashed curve) is the original (unmodified) version of the gene, and CAI, LAI-D, TDA-R, TDR-D, and TDR-R arc modified versions of mCherry gene. (17B) Bar graph of fold change in bacterial growth rates of each ORF version relative to the growth rates in mCherry. (17C) Same as in 17B but calculated for the average maximal density. (17D) Bar graph of fold of growth rates in B. subtilis relative to E. coli.

FIGS. 18A-D: (18A) Representative fluorescence intensity plots of all ORF variants in B. subtilis (top) and in E. coli (bottom). Note that the control lacked the mCherry gene, and thus didn't exhibit fluorescence, and served for background subtraction. (18B) Bar graph of fold change in average maximal fluorescence intensity of each ORF version relative to mCherry. (18C) The same as in 18B but calculated for the average normalized fluorescence. (18D) Bar graph of fold of average normalized fluorescence in B. subtilis relative to E. coli.

FIG. 19: A schematic of a method of fusion PCR to link a plasmid to its bacterial host. A set of forward and reverse primers are used to amplifying the GOI, wherein the primers include an appended tail that targets this bacteria's 16S rRNA gene. GOI amplicon serves as a forward primer in 16S rRNA gene amplification, which results in a fused amplicon product that can be further quantified via qPCR.

### DETAILED DESCRIPTION OF THE INVENTION

The present invention, in some embodiments, provides methods for engineering a nucleic acid molecule comprising a coding region optimized for expression in a first organism and deoptimized for expression in a second organism.

The invention is based, at least in part, on the surprising findings stemming from a different view of the biological process, in which each genetic element that is linked to gene expression is examined and synthetically altered, instead of working with genetic building blocks as given. This method is generic and computational, aiming to fit selected genetic information to a given microbiome, by modulating expression in wanted and unwanted hosts of the modification. For instance, in the case of the human gut microbiome, some bacteria are symbiotic- and others are pathogenic. An effective community engineering process would likely target a subgroup of the pathogenic bacterial species which can be viewed as the wanted hosts of the modification in this case (which can include for example a gene that decreases their growth rate); however, it should probably avoid expression in the symbiotic bacteria as much as possible, which can be defined as the unwanted hosts.

This approach is designed by considering the effects of horizontal gene transfer (HGT) on the genetic construct and interactions it facilitates. Additionally, this method takes into account the various degrees of characterizations that can exist for a certain microbiome and can function even with very minimal metagenomic information (our current implementation uses annotated genomes and can potentially be used with metagenomically assembled genomes correspondingly). Lastly, this method is designed to modify the microbiome for longer time periods. It is relatively resistant to the environmental damage of the genetic information, as each genetic element is examined and treated individually. The design process considers the fitness effect of the modification on its proposed hosts and modulates the burden it poses accordingly.

The current design approach deals with the three main processes related to gene expression: entry into the cell, transcription, and translation. First, entry into the bacterial cell is modulated by editing the presence of restriction sites, increasing chances of digestion upon entry of the plasmid into an unwanted host compared to a wanted host. Similarly, uptake signal sequences (USS) optimization also provides modulation at this step. Next, the transcription process is optimized by discovery of genetic motifs which are likely linked to TFs which are present explicitly in the wanted hosts and are related to transcription initiation. Lastly, the translation process includes re-coding of the ORF based on translation efficiency modulation by exploitation of the degree of freedom posed by the redundancy of the genetic code.

In order to test our models, we performed both in-silico and in-vitro tests. The in-vitro tests have shown the effect of the optimization process on the expression rate of a selected protein, which was both higher for the wanted host (B. subtilis) and lower for the unwanted host (E. coli) simultaneously. Moreover, the attached fitness effect is just as important—while the modified sequence does not pose a significant burden compared to the initial sequence in the wanted host, this cannot be said for the presence of the plasmid in the unwanted hosts.

The clear fitness decrease in the unwanted hosts caused by the optimization process might not have that much of a significant effect in lab conditions, however the meaning of this change is that when dealing with an actual microbiome, there will be a stronger evolutionary pressure against existence of the plasmid in the unwanted hosts, thus further propagating the designed expression differentiation in the microbiome. The in-silico analysis supplied complementary views on each one of the computational methods individually, with the scale up process (from two organisms to an entire microbiome) defining the relevance of the different tests.

In some embodiments, the method is an in vitro method. In some embodiments, the method is an ex vivo method. In some embodiments, the method is a computerized method. In some embodiments, the method is a method for producing an optimized nucleic acid molecule. In some embodiments, the method is a method for optimizing a nucleic acid molecule. In some embodiments, the method is a method for engineering a nucleic acid molecule comprising an optimized coding region. In some embodiments, optimized is optimized for expression. In some embodiments, optimized is optimized for transcription. In some embodiments, optimized is optimized for translation. In some embodiments, expression is mRNA expression. In some embodiments, expression is protein expression. In some embodiments, optimized is optimized for the first organism. In some embodiments, optimized is deoptimized for the second organism. In some embodiments, optimized is optimized for expression in the first organism and deoptimized for expression in the second organism.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infections origin, amplification products, modified nucleic acids, plasmidical or organellar nucleic acids and artificial nucleic acids such as oligonucleotides. In some embodiments, the nucleic acid molecule is a polynucleotide molecule. In some embodiments, the nucleic acid molecule is a DNA molecule. In some embodiments, the nucleic acid molecule is an RNA molecule.

As used herein, the term “encoding” refers to molecule comprising a DNA sequence which can be transcribed into an RNA sequence which can be translated into the encoded protein or a molecule comprising the RNA sequence which can be translated into the encoded protein. In some embodiments, the molecule is a DNA molecule. In some embodiments, the molecule is an RNA molecule. In some embodiments, the DNA is cDNA. In some embodiments, the molecule is a DNA/RNA hybrid. In some embodiments, the molecule comprises non-naturally occurring nucleotides.

In some embodiments, the nucleic acid molecule is a plasmid. In some embodiments, the nucleic acid molecule is a vector. In some embodiments, the vector is an expression vector. In some embodiments, the vector is configured for expression of the coding region.

Expressing of a gene within a cell is well known to one skilled in the art. It can be carried out by, among many methods, transfection, viral infection, or direct alteration of the cell's genome. In some embodiments, the nucleic acid molecule is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives. In some embodiments, the vector is a bacterial expression vector. Examples of bacterial expression vectors include, but are not limited to pACYC177, pASK75, pBADM, pUC, pBR322, pGAT, pMal, ColE1, p15H, and pZA31, to name but a few. These vectors are commercially available from companies such as Invitrogen, Promega, Strategene, Clonthech, Novagen, Sigma, Life Technologies and New England Biolabs.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the organism is a bacterium. In some embodiments, the organism is a prokaryotic organism. In some embodiments, the organism is a eukaryotic organism. In some embodiments, the organism is a single celled organism. In some embodiments, the organism is a virus. In some embodiments, the organism is not a virus. In some embodiments, the organism is a yeast. In some embodiments, the organism is a fungus.

In some embodiments, the first organism is a desired organism. In some embodiments, the second organism is an undesired organism. In some embodiments, the first organism is a target organism. In some embodiments, the second organism is an off-target organism. In some embodiments, the first and second organisms are found in the same habitat. In some embodiments, the first and second organism are found in the same microenvironment. In some embodiments, there is horizontal gene transfer between the first organism and the second organism. In some embodiments, the molecule is designed for expression in the first organism and not the second organism. In some embodiments, the molecule is configured for expression in the first organism and not the second.

In some embodiments, the first organism is a first set of organisms. In some embodiments, the second organism is a second set of organisms. In some embodiments, a set is a plurality of organisms. In some embodiments, a set is at least 2, 3, 4, 5, 6, 7, 8, 9 or 10 organisms. Each possibility represents a separate embodiment of the invention. In some embodiments, a set is at least 2 organisms. In some embodiments, a set is at least 3 organisms. In some embodiments, the first set and the second set are mutually exclusive. In some embodiments, the first set is a first class of organisms, and the second set is a second class of organisms. In some embodiments, organisms in a set are related. In some embodiments, organisms in a set carry out horizontal gene transfer between them. In some embodiments, organisms in a set all share a common property. In some embodiments, the first and second set of organisms are comprised in a biological sample. In some embodiments, the first and second set of organisms coexist in a biological sample. In some embodiments, the biological sample is soil. In some embodiments, the biological sample is from a mammalian organism. In some embodiments, the mammal is a human. In some embodiments, the sample is a gut microbiome sample. In some embodiments, the first and second set of organisms live in a microbiome. In some embodiments, the first and second set of organisms live in sufficient proximity to each other so as to allow horizontal gene transfer.

### Translational Optimization

By a first aspect, there is provided a method of for engineering a nucleic acid molecule comprising a coding region, the method comprising calculating codon usage in a first organism and codon usage in a second organism and replacing at least one codon of a nucleotide sequence of the coding region with a synonymous codon, wherein the synonymous codon is selected for in the first organism based and deselected for in the second organism, thereby engineering a nucleic acid molecule.

In some embodiments, the molecule comprises at least one coding region. In some embodiments, the molecule comprises a plurality of coding regions. In some embodiments, the coding region comprises a nucleotide sequence. In some embodiments, the molecule comprises at least one coding sequence. In some embodiments, the nucleotide sequence is the coding sequence. In some embodiments, the nucleotide sequence is a portion of the coding region. In some embodiments, the molecule comprises a plurality of coding sequences. In some embodiments, the molecule comprises a plurality of nucleotide sequences. In some embodiments, a portion is at least 5, 10, 15, 20, 25, 30, 35, 40, 45, 50, 55, 60, 65, 70, 75, 80, 85, 90, 95, 97, 99 or 100% of the coding region. Each possibility represents a separate embodiment of the invention. In some embodiments, a portion is at least 3, 6, 9, 12, 15, 18, 21, 24, 27, 30, 33, 36, 39, 42, 45, 48, 51, 54, 57, 60, 90, 120, 150, 180, 210, 240, 270, 300, 330. 360. 390. 420. 450. 480, 510, 540, 570 or 600 nucleotides. Each possibility represents a separate embodiment of the invention. In some embodiments, a portion is at most all of the coding region.

In some embodiments, the coding region encodes for a protein of interest. In some embodiments, the coding region is a gene of interest. In some embodiments, the coding region is a DNA encoding the protein of interest. In some embodiments, the coding region is an RNA translatable to the protein of interest. In some embodiments, the coding region comprises a coding sequence mutated to optimize its expression. In some embodiments, the coding region comprises a coding sequence comprising at least one mutation that optimizes its expression. In some embodiments, the coding sequence is a naturally occurring coding sequence. In some embodiments, the coding sequence is a wild-type coding sequence. In some embodiments, the coding sequence is an endogenous coding sequence. In some embodiments, the coding sequence is an exogenous coding sequence. In some embodiments, the protein of interest is not expressed by the first organism. In some embodiments, the protein of interest is not expressed by the second organism. In some embodiments, the protein of interest is a heterologous transgene.

In some embodiments, the coding sequence is optimized. In some embodiments, the optimizing comprises mutating the sequence. In some embodiments, the optimized sequence is a non-naturally occurring sequence. In some embodiments, a non-naturally occurring sequence comprises at least one mutation. In some embodiments, the mutation is a mutation of a naturally occurring sequence.

In some embodiments, the optimized sequence comprises at least 1, 2, 3, 4, 5, 6, 7, 8, 9, 10, 12, 15, 17, 20, 25, 30, 35, 40, 45, 50, 60, 70, 75, 80, 90 or 100 mutations. Each possibility represents a separate embodiment of the invention. In some embodiments, the optimized sequence comprises at least 1 mutation. In some embodiments, the mutation is a synonymous mutation. In some embodiments, the mutation does not change the amino acid sequence encoded by the coding region. As used herein, the term “synonymous mutation” refers to a mutation that does not alter the amino acid sequence encoded by the nucleotide sequence. In some embodiments, the mutation results in the replacement of the at least one codon with the synonymous codon. In some embodiments, the optimized sequence comprises at least 1, 2, 3, 4, 5, 6, 7, 8, 9, 10, 12, 15, 17, 20, 25, 30, 35, 40, 45, 50, 60, 70, 75, 80, 90 or 100 codons replaced with synonymous codon. Each possibility represents a separate embodiment of the invention. In some embodiments, the optimized sequence comprises at least 1 codon replaced with a synonymous codon. One skilled in the art will be able to determine based on the first and second organisms the minimum number of codons to be substituted. In some embodiments, protein expression in the first and second organisms after substitution can be measured and compared to protein expression without substitutions to determine if a sufficient number of codons have been substituted. In some embodiments, all codons of the nucleotide sequence that can be, are replaced with a synonymous codon selected for in the first organism. In some embodiments, all codons of the nucleotide sequence that can be, are replaced with a synonymous codon deselected for in the second organism. In some embodiments, all codons of the nucleotide sequence that can be, are replaced with a synonymous codon selected for in the first organism and deselected from in the second organism.

The term “codon” refers to a sequence of three DNA or RNA nucleotides that correspond to a specific amino acid or stop signal during protein synthesis. The codon code is degenerate, in that more than one codon can code for the same amino acid. Such codons that code for the same amino acid are known as “synonymous” codons. Thus, for example, CUU, CUC, CUA, CUG, UUA, and UUG are synonymous codons that code for Leucine. Synonymous codons are not used with equal frequency. In general, the most frequently used codons in a particular cell are those for which the cognate tRNA is abundant, and the use of these codons enhances the rate and/or accuracy of protein translation. Conversely, tRNAs for rarely used codons are found at relatively low levels, and the use of rare codons is thought to reduce translation rate and/or accuracy. “Codon bias” as used herein refers generally to the non-equal usage of the various synonymous codons, and specifically to the relative frequency at which a given synonymous codon is used in a defined sequence or set of sequences.

Synonymous codons are provided in Table 1. The first nucleotide in each codon encoding a particular amino acid is shown in the left-most column; the second nucleotide is shown in the top row; and the third nucleotide is shown in the right-most column.

TABLE 1
Genetic code

| | U | C | A | G | |
|---|---|---|---|---|---|
| U | Phe | Ser | Tyr | Cys | U |
| | Phe | Ser | Tye | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

In some embodiments, greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, or 100% of all codons in the coding sequence have been substituted. Each possibility represents a separate embodiment of the present invention.

In some embodiments, greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, or 100% of codons that have synonymous codons with different frequencies in first and second organism have been substituted. Each possibility represents a separate embodiment of the present invention. In some embodiments, a plurality of codons having synonymous codons with different frequencies have been substituted. In some embodiments, a plurality of codons having synonymous codons with higher frequencies have been substituted. In some embodiments, a plurality of codons having synonymous codons with lower frequencies have been substituted. In some embodiments, higher is higher in the second organism than the first. In some embodiments, higher is higher in the first organism than the second. In some embodiments, lower is lower in the second organism than the first. In some embodiments, lower is lower in the first organism than the second. It will be understood that to optimize a coding sequence for expression in one organism and not the other the codons with highest frequency in the first organism will be selected and codons with highest frequency in the second organism will be deselected. If a codon is already the most frequent codon in the first organism, then no substitution should be made. Similarly, if a codon is already the least frequent codon in the second organism, then no substitution should be made.

In some embodiments, optimized is codon optimized. In some embodiments, the codon bias is optimized. In some embodiments, calculating codon usage comprises calculating codon usage bias (CUB). In some embodiments, codon bias is optimized to match the codon bias in the first organism. In some embodiments, codon bias is optimized to not match the codon bias in the second organism. In some embodiments, codon optimized comprises codon usage bias (CUB) optimization. In some embodiments, the CUB is codon bias. In some embodiments, CUB optimization comprises tRNA adaptation index (tAI) optimization. In some embodiments, CUB optimization is by tAI. In some embodiments, CUB optimization comprises codon adaptation index (CAI) optimization. In some embodiments, CUB optimization is by CAI. In some embodiments, CUB optimization comprises typical decoding rate (TDR) optimization. In some embodiments, CUB optimization is by TDR. Performance of CUB, tAI, CAI, TDR and other algorithmic optimizations are well known in the art and are further described hereinbelow. A skilled artisan with a target organism, coding sequences of genes expressed in the target organism and expression levels of those sequences in the target organism can calculate the indexes and biases recited herein. Thus, optimization may include replacing a given codon in the codon region by a synonymous but more frequently used codon in the first organism or a synonymous but less frequently used codon in the second organism. In some embodiments, the frequency is calculated by tAI. In some embodiments, the frequency is calculated by CAI. In some embodiments, the frequency is calculated by TDR. In some embodiments, calculation is relative to null model. In some embodiments, the null model is a VCUB null model. Methods of generating and analyzing these null models are well known in the art.

In some embodiments, the synonymous codon is selected for in the first organism. In some embodiments, the synonymous codon is deselected from in the second organism. In some embodiments, the synonymous codon is selected for in the first organism and deselected for in the second organism. In some embodiments, the selection is based on the CUB in the first organism. In some embodiments, the deselection is based on the CUB in the second organism. In some embodiments, the CUB is the calculated CUB. In some embodiments, the CUB is calculated based on tAI, CAI, or TDR.

Calculating the relative frequency of a codon in a gene or a set of genes is understood to refer to counting the number of times a codon is used in that gene or set of genes and counting how many times codons synonymous to that codon are used. Dividing the number of times a codon is used over the total number of codons that code for the same amino acid as that codon gives the relative frequency of that codon. For a non-limiting example, if the codon UUU (coding for Phe) appears 5 times in the set of gene, and UUC (the only synonymous codon to UUU) appears 15 times, then the frequency of codon UUU is 25%.

In some embodiments, the frequency of usage is the relative synonymous codon frequency. The term “relative synonymous codons frequencies” as used herein refers to the frequency at which a codon is used relative to other synonymous codons within a specific reference set. Relative synonymous codons frequencies can be represented as a vector which entries correspond to each one of 61 coding codons (stop codons are excluded):

$$\begin{matrix}RSCF = ( RSCF [ 1 ] , … , RSCF [ 61 ] ) \\ RSCF [ i ] = \frac{{q}_{i}}{{\sum }_{j ∈ syn [ i ]} {q}_{j}} , {{\sum }_{j ∈ syn [ i ]}}_{} RSCF [ j ] = 1\end{matrix}$$

where qᵢ is the number of appearances of codon i in a sequence, syn[i] is a subset of indexes in RSCF pointing at codons synonymous to codon i.

In some embodiments, the tAI is the relative codon-tRNA adaptation index. The term “relative codon-tRNA adaptation” as used herein refers to how well a codon is adapted to the tRNA pool relative to other synonymous codons within a specific reference set. The tRNA pool in a cell can change over time depending on the cellular context. In some embodiments, the tRNA pool is different between the first organism and the second organism.

Relative codon-tRNA adaptation and the tRNA adaptation index (tAI) quantify the adaptation of one codon, or a coding region, respectively, to the tRNA pool. Let tCGNi j be the copy number of the j-th anti-codon that recognizes the i-th codon and let Si j be the selective constraint of the codon-anti-codon coupling efficiency. The S vector [sI:U, sG:C, SU:A, sC:G, sG:U, sI:C, sI:A, sU:G, sL:A] was defined for E. coli as [0, 0, 0, 0, 1, 0.25, 0.81, 1, 0.71] according to optimization performed previously (Sabi R, et al., DNA Research, 2014, 21:511-525). Thus, the absolute adaptiveness value of a codon of type i (1≤i≤61; stop codons are excluded) to the tRNA pool is defined by:

$${W}_{i} = {\sum }_{j = 1}^{{n}_{i}} ( 1 - {S}_{ij} ) {tCGN}_{ij}$$

For each amino acid, the weight of each of its codons, is computed as the ratio between the absolute adaptiveness value of the codon and the maximal absolute adaptiveness value of the synonymous codons for that amino acid:

$${w}_{i} = \frac{{W}_{i}}{{max}_{j ∈ syn [ i ]} {W}_{j}}$$

where Wᵢ is the absolute adaptiveness of codon i in a sequence, syn[i] is a subset of indexes in pointing at codons synonymous to codon i. wᵢ takes values from 0 (not adapted) to 1 (maximally adapted). If the weight value is zero a value of 0.5 is used. tAI is the geometric mean of wᵢ (relative codon-tRNA adaptation) over codons of a coding sequence.

In some embodiments, optimizing codons comprises optimizing the expression levels of the sequence (s) with respect to the codons Typical Decoding Rate (TDR) in the first nd second organism basing on available ribosomal profiling data. To estimate TDR, a statistical mode, which takes into consideration the skewed nature of the ribose read count distribution can be used. This model describes the readcount histogram of each codon as an output of a random variable which is a sum of two random variables: a normal and an exponential variable. Thus, the distribution of this new random variable includes three parameters and is called EMG distribution. In this model, the typical codon decoding time was described by the normal distribution with two parameters: mean (μ) and standard deviation [symbol] ; the μ parameter represents the location of the mean of the theoretical Gaussian component that should be obtained if there are no phenomena such as pauses/biases/ribosomal traffic jams; σ represents the width of the Gaussian component. The exponential distribution has one parameter λ which represents the skewness of the readcount distribution due to reasons such as ribosomal jamming caused by codons with different decoding times, extreme pauses, incomplete halting of the ribosomes, biases in the experiment, etc. The EMG is defined as follows:

$$\begin{matrix}f ( x ; μ , σ , λ ) = \frac{λ}{2} {e}^{\frac{λ}{2} ( 2 μ + {λσ}^{2} - 2 x )} erfc ( \frac{μ + {λσ}^{2} - x}{\sqrt{2} σ} ) , \\ erfc ( x ) = 1 - erf ( x ) = {\int }_{x}^{∞} {e}^{- {t}^{2}} dt\end{matrix}$$

These three parameters may be estimated for each codon at different replication stages based on time dependent ribosome profiling data by fitting the suggested model to the given read count distribution (e.g., using the maximal likelihood estimation or any other algorithm). 1/μ is defined to be the Typical Decoding Rate (TDR) of each codon.

In some embodiments, optimization comprises synonymous substitution with the optimal codon. In some embodiments, the optimal codon is the codon with the lowest loss score. In some embodiments, the loss score is calculated by a loss function. In some embodiments, the loss function comprises the ratio of loss, or loss ratio (R). In some embodiments, the loss function comprises the difference lost or loss difference (D). In some embodiments, the optimization is a CUB optimization. In some embodiments, the optimization is a tAI-R optimization. In some embodiments, the optimization is a tAI-D optimization. In some embodiments, the optimization is a TDR-R optimization. In some embodiments, the optimization is a TDR-D optimization.

In some embodiments, optimized is optimized in all organisms of the first set. In some embodiments, deoptimized is deoptimized in all organisms of the second set. In some embodiments, within the organism of the first set for which the ORF is least optimized and within the organism of the second set for which the ORF is least deoptimized the ORF is still more optimized in the organism of the first set. In some embodiments, more optimized is more highly expressed. In some embodiments, more optimized is produces a better growth rate. In some embodiments, an optimization score is calculated for each organism. In some embodiments, a nucleic acid molecule with a score beyond a predetermined threshold is considered optimized/deoptimized. In some embodiments, a nucleic acid molecule with a statistically significant score is considered optimized/deoptimized.

In some embodiments, the method simultaneously optimizes for the first organism and deoptimizes for the second organism. In some embodiments, the method produces the greatest optimization in the first organism and the greatest deoptimization in the second organism. In some embodiments, more than one method of optimization/deoptimization is calculated and the method that produces the greatest difference from the optimized organism to the deoptimized organism is selected. In some embodiments, the difference is difference in ORF expression. In some embodiments, expression is protein expression. In some embodiments, expression is mRNA expression. In some embodiments, the difference is difference is organism survival. In some embodiments, the difference is difference is organism growth rate.

### Transcriptional Optimization

By another aspect, there is provided a method of for engineering a nucleic acid molecule comprising a coding region, the method comprising receiving a first list of sequences of regulatory elements from the first organism and a second list of regulatory elements in the second organism, selecting sequence motifs enriched in the first list and/or depleted in the second list, engineering a regulatory element comprising a plurality of selected sequence motifs and operably linking the engineered regulatory element to the coding region, thereby engineering a nucleic acid molecule.

By another aspect, there is provided a method of for engineering a nucleic acid molecule comprising a coding region, the method comprising receiving a first list of sequences of regulatory elements from the first organism and a second list of regulatory elements in the second organism, selecting sequence motifs enriched in the second list and/or depleted in the first list, engineering a regulatory element comprising a plurality of selected sequence motifs and operably linking the engineered regulatory element to the coding region, thereby engineering a nucleic acid molecule.

In some embodiments, the list comprises at least 2, 3, 4, 5, 6, 7, 8, 9, 10, 11, 12, 13, 14, 15, 20, 25, 30, 35, 40, 45, 50, 60, 70, 75, 80, 90 or 100 sequences. Each possibility represents a separate embodiment of the invention. In some embodiments, the regulatory element is a positive regulatory element. In some embodiments, the regulatory element regulates transcription of the coding sequence. In some embodiments, the regulatory element drives transcription of the coding sequence. In some embodiments, the regulatory element is a promoter. In some embodiments, the regulatory element is an enhancer. In some embodiments, the regulatory element is an activator.

In some embodiments, the regulatory elements are from highly expressed gene. In some embodiments, the highly expressed genes are highly expressed in the first organism. In some embodiments, highly expressed comprises the top 1, 5, 7, 10, 15, 20, 25, 30, 35, 40 45 or 50% of expressed genes. Each possibility represents a separate embodiment of the invention. In some embodiments, highly expressed comprises the most highly expressed 1, 5, 7, 10, 15, 20, 25, 30, 35, 40 45 or 50% of genes. Each possibility represents a separate embodiment of the invention. In some embodiments, highly expressed genes do not comprise the most highly expressed and second most highly expressed genes. In some embodiments, highly expressed is the top 10% most highly expressed. In some embodiments, highly expressed is the top 20% most highly expressed. In some embodiments, highly expressed is the top 30% most highly expressed. In some embodiments, highly expressed is expressed above a predetermined threshold. In some embodiments, highly expressed based on a predetermined threshold percentage of genes. In some embodiments, the first list comprises regulatory elements from highly expressed genes of the first organism. In some embodiments, the second list comprises regulatory elements from highly expressed genes of the second organism.

In some embodiments, a sequence motif comprises at least 4, 5, 6, 7, 8, 9, 10, 11, or 12 nucleotides. Each possibility represents a separate embodiment of the invention. In some embodiments, a sequence motif comprises at most 10, 12, 14, 15, 17, 18, 20, 25, 30, 35, 40, 45, 50, 60, 70, 75, 80, 90, 100, 150, 200, 250, 300, 400 or 500 nucleotides. Each possibility represents a separate embodiment of the invention. In some embodiments, a motif is a sequence which produces a regulatory effect. In some embodiments, a motif is a transcription factor binding site.

In some embodiments, the selecting is selecting sequence motifs enriched in the first list. In some embodiments, the selecting is selecting sequence motifs depleted in the second list. In some embodiments, the selecting is selecting sequence motifs enriched in the first list and depleted in the second list. In some embodiments, the method further comprises receiving expression data from the first organism and second organism and selecting highly expressed genes. In some embodiments, the method further comprises selecting regulatory sequences from the highly expressed genes. In some embodiments, the highly expressed genes are inferred based on CUB rankings of coding sequences of all genes in the organism. In some embodiments, expression data is not available for an organism and the highly expressed genes are inferred based on CUB rankings of coding sequences of all genes in the organism.

Motif identification may be done by any method known in the art or any algorithm known in the art. In some embodiments, the STREME software is used for motif identification. In some embodiments, selecting comprises employing a Markov model. In some embodiments, the Markov model is a hidden Markov model. In some embodiments, the hidden Markov model comprise 3 hidden layers. In some embodiments, the Markov model is a k−1 order Markov model. Methods of employing such a model are well known in the art and are described hereinbelow.

In some embodiments, a motif is a transcription enhancing motif. In some embodiments, the motif in the first organism is a transcription enhancing motif. In some embodiments, a transcription enhancing motif is a motif that regulates transcription. In some embodiments, the motif is a promoter motif. In some embodiments, the motif is enriched in promoters. In some embodiments, enriched is as compared to non-promoter sequence. In some embodiments, enriched is as compared to intragenic sequence. In some embodiments, a transcription enhancing motif is a motif enriched in promoters as compared to intragenic sequence. In some embodiments, the transcription enhancing motif is enriched in promoters of a wanted organism as compared to intragenic regions of the wanted organism. In some embodiments, a motif is a transcription decreasing motif. In some embodiments, the motif in the second organism is a transcription decreasing motif. In some embodiments, a transcription decreasing motif is an anti-motif. In some embodiments, the transcription decreasing motif is enriched in intragenic regions of an unwanted organism as compared to promoters of the unwanted organism.

In some embodiments, motifs from the first organism are selected. In some embodiments, anti-motifs from the second organism are selected. In some embodiments, the selected motifs and anti-motifs are in a regulatory element linked to the open reading frame. In some embodiments, the selected motifs and anti-motifs are operatively linked to the open reading frame. In some embodiments, motifs from the second organism are selected. In some embodiments, anti-motifs from the first organism are selected. In some embodiments, the selected motifs and anti-motifs are removed from a regulatory element linked to the open reading frame. In some embodiments, the selected motifs and anti-motifs are excluded from the design of a regulatory element to be linked to the open reading frame. In some embodiments, when generating motifs/anti-motifs in a regulatory element, mismatches between mapped motifs/anti-motifs and promoters are alternated.

In some embodiments, the engineering comprises linking selected sequence motifs. In some embodiments, linking is directly linking. In some embodiments, linking is via a nucleotide linker. In some embodiments, the linker comprises at least 1, 2, 3, 4, 5, 6, 7, 8, 9 or 10 nucleotides. Each possibility represents a separate embodiment of the invention. In some embodiments, the linker comprises at most 5, 10, 15, 20, 25, 30, 35, 40, 45 or 50 nucleotides. Each possibility represents a separate embodiment of the invention. In some embodiments, the linker is a repetitive sequence. In some embodiments, the linker is nonstructured.

In some embodiments, the engineered regulatory element is an artificial regulatory element. In some embodiments, artificial is non-natural. In some embodiments, artificial is not occurring in nature. In some embodiments, the artificial regulatory element comprises a plurality of selected motifs. In some embodiments, the artificial regulatory element comprises at least 2, 3, 4, 5, 6, 7, 8, 9 or 10 of selected motifs. Each possibility represents a separate embodiment of the invention. In some embodiments, motifs are transcription factor binding sites. In some embodiments, the motifs are ordered. In some embodiments, the motifs are unordered. In some embodiments, the order is the same as the order found in the highly expressed genes. In some embodiments, the order is based on the order found in the highly expressed genes.

In some embodiments, engineering comprises selected an endogenous regulatory element. In some embodiments, the endogenous regulatory element is from the first list. In some embodiments, the endogenous regulatory element is enriched for the selected sequence motifs. In some embodiments, the endogenous regulatory element is depleted for the selected sequence motifs. In some embodiments, enriched is highly enriched. In some embodiments, depleted is highly depleted. In some embodiments, the method comprises ranking the regulatory elements from the first list. In some embodiments, the ranking is based on their enrichment with the selected sequence motifs. In some embodiments, the ranking is based on their depletion of motifs from the second list. In some embodiments, the significance of enrichment is scored. In some embodiments, each motif in the first list is scored for significance of enrichment in the first list. In some embodiments, the ranking of sequences from the first list is based on their enrichment and the significance of enrichment. In some embodiments, highly enriched is within the top 1, 3, 5, 7, 10, 15, 20 or 25% of ranked sequences. Each possibility represents a separate embodiment of the invention. In some embodiments, the ranking employs a k−1 order Markov model.

In some embodiments, the method further comprises producing at least one mutation in an endogenous regulatory element. In some embodiments, the mutation produces at least one selected sequence motif. In some embodiments, the mutation abolishes at least one sequence motif enriched in the second list. In some embodiments, an artificial regulatory element comprises at least 1, 2, 3, 4, 5, 6, 7, 8, 9 or 10 mutations. Each possibility represents a separate embodiment of the invention.

In some embodiments, MAST is used to align the motifs to the promoter. In some embodiments, plurality of promoters is aligned. In some embodiments, the engineered promoter that produces the highest expected value of optimization is selected. In some embodiments, the expected value is based on the initial significance of the motif and the quality of the alignment. In some embodiments, preexisting promoter is selected due to the presence of desired motifs and the absence of undesired motifs. In some embodiments, a promoter is engineered to contain desired motifs and lack undesired motifs.

In some embodiments, the coding sequence is operably linked to at least one regulatory element. The term “operably linked” is intended to mean that the nucleotide sequence of interest is linked to the regulatory element or elements in a manner that allows for expression of the nucleotide sequence. In some embodiments, the engineered regulatory element is operably linked to the coding region. In some embodiments, nucleic acid molecule is configured such that the regulatory element is operably linked to the coding sequence.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. In some embodiments, the promoter comprises the first 200 bases upstream of the ORF. In some embodiments, the promoter consists of the first 200 bases upstream of the ORF. In some embodiments, the promoter is the core promoter.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA. Prokaryotes use the same RNA polymerase to transcribe all of their genes. Prokarytotic polymerase has multiple subunits, often delineated as alpha, alpha, beta, beta prime and omega.

### Restriction Enzyme Target Optimization

By another aspect, there is provided a method of for engineering a nucleic acid molecule comprising a coding region, the method comprising determining target sequences of cleaving agents expressed by the first organism and target sequences of cleaving agents expressed by the second organism and altering a sequence of the nucleic acid molecule to include at least one of the target sequences expressed by the second organism or to remove at least one target sequence expressed by the first organism, thereby engineering a nucleic acid molecule.

In some embodiments, the cleaving agents are nucleic acid molecule cleaving agents. In some embodiments, the cleaving agents are DNA cleaving agents. In some embodiments, the cleaving agents are RNA cleaving agents. In some embodiments, the DNA cleaving agent is a restriction enzyme. In some embodiments, the restriction enzyme is a palindromic restriction enzyme. Restriction enzymes are well known in the art and the target sequences which they cut are also well know. Lists and their targets can be found in a variety of databases and well as commercial sites selling the enzymes, such as for example REBASE (re3data.org).

In some embodiments, the altering comprises producing at least one target sequence of a restriction enzyme expressed by the second organism. In some embodiments, expressed is only expressed. In some embodiments, the target sequence is a palindromic target sequence. In some embodiments, the altering comprises removing a target sequence of a restriction enzyme expressed by the first organism. In some embodiments, removing is deleting. In some embodiments, removing is mutating. Restriction enzymes are very sequence specific, and a single nucleotide mutation can abolish the binding and cutting of the restriction enzyme. In some embodiments, overlapping target sequences are not generated. In some embodiments, one of a plurality of overlapping target sequences are selected for production in the molecule. In some embodiments, selection comprises selecting the target sequence found in the most organism of the second set. In some embodiments, selection comprises selecting the target sequence found in an organism of the second set with the fewest number of target sequences that can be generated in the molecule. It will be understood by a skilled artisan that there is a desire to exclude expression in all of the organisms of the second set and so when selecting from overlapping sequences the ones from the hard to target organisms will be chosen. In some embodiments, one of a plurality of overlapping target sequences are selected for removal from the molecule. In some embodiments, selection comprises selecting the target sequence found in the most organism of the first set.

In some embodiments, target sequences are of cleaving agents only expressed by the first organism. In some embodiments, target sequences are of cleaving agents only expressed by the second organism. In some embodiments, the altering produces at least one target sequence of a cleaving agent expressed only in the second organism and not in the first organism. In some embodiments, the altering erases at least one target sequence of a cleaving agent expressed only in the first organism and not in the second organism. In some embodiments, the altering erases at least one target sequence of a cleaving agent expressed in the first organism.

In some embodiments, the cleaving agent is a cleaving protein. In some embodiments, the cleaving agent is a ribozyme. In some embodiments, the cleaving agent is a cleaving ribo-protein complex. In some embodiments, the cleaving agent is a nuclease. In some embodiments, the cleaving agent is a nickase. In some embodiments, the cleaving agent is genome editing protein. In some embodiments, a genome-editing protein is selected from the group consisting of a clustered Regularly Interspaced Short Palindromic Repeats (CRISPR)-associated nuclease, a Zinc-finger nuclease (ZFNs), a meganuclease and a transcription activator-like effector nuclease (TALEN). In some embodiments, the genome-editing protein is a meganuclease. In some embodiments, the genome-editing protein is a natural meganuclease. In some embodiments, the genome-editing protein is a modified/engineered meganuclease.

In some embodiments, the genome-editing protein is a CRISPR-associated protein. In some embodiments, the CRISPR-associated protein is CRISPR-associated protein 9 (Cas9). In some embodiments, the CRISPR-associated protein is Cas9 or a Cas9 ortholog. In some embodiments, the CRISPR-associated protein is Cas9 or a Cas9 variant. In some embodiments, the CRISPR-associated protein is Cas9 or a Cas9 homolog. Other CRISPR-associated proteins are well known in the art and may be employed, such as for example CSF1, Cas12a, Cas13a, CasI, CasIB, Cas2, Cas3, Cas5, Cas6, Cas7, Cas8, Cas100, Csy1, Csy2, Csy3, Cse1, Cse2, Csc1, Csc2, Csa5, Csm2, Csn2, Csm3, Csm4, Csm5, Csm6, Cmr1, Cmr4, Cmr5, Cmr6, Csb1, Csb2, Csb3, Csx14, Csx17, Csx10, Csx6, CsaX, Csx3, Csx15, Csf1, Csf2, Csf3, Csf4, PE1, PE2, PE3, and MAD7.

In some embodiments, the altering is done in a coding region. In some embodiments, the altering does not change the amino acid sequence encoded by the coding region. In some embodiments, the altering produces a synonymous mutation. In some embodiments, two alterations are made flanking a coding region. In some embodiments, an alteration is made 5′ to a coding region and an alteration is made 3′ to a coding region. In some embodiments, the altering is in a regulatory region. In some embodiments, a regulatory region is a regulatory element. In some embodiments, the regulatory region is one required for expression of the coding region. In some embodiments, the regulatory region is one that enhances expression of the coding region. In some embodiments, the regulatory region is an essential regulatory region. In some embodiments, the altering is done in an essential region of the nucleic acid molecule. In some embodiments, an essential region is selected from the coding region, a regulatory region, an origin of replication and an uptake signal sequences. In some embodiments, the altering is done anywhere in the molecule. It will be understood by a skilled artisan that as cutting will de-circularize a plasmid it may be sufficient to inhibit expression and/or transfer. Further, should recircularization occur, if a portion or all of a coding region has been removed it will negatively impact the survival/growth of the second organism.

In some embodiments, the altering comprises producing a PAM sequence of a CRISPR protein of the second organism. In some embodiments, the altering comprises producing a spacer sequence expressed by the second organism. In some embodiments, expressed by is expressed only by. In some embodiments, altering comprises inserting the spacer sequence downstream of a PAM. In some embodiments, the PAM sequence is already present in the nucleic acid molecule and the altering comprises inserting the spacer sequence in proper frame to the PAM sequence. In some embodiments, the altering comprises producing the PAM and the spacer sequence. In some embodiments, the PAM and spacer sequence are produced in proper frame to teach other. In some embodiments, proper frame is the proper distance such that the CRISPR protein will cut the spacer sequence.

In some embodiments, the method comprises altering a sequence of the nucleic acid molecule to include at least one of the target sequences expressed by the second organism and to remove at least one target sequence expressed by the first organism. In some embodiments, after including at least one of the target sequences expressed by the second organism a check is performed to ensure a target sequence expressed by the first organism hasn't been created. In some embodiments, altering a sequence of the nucleic acid molecule to include at least one of the target sequences expressed by the second organism does not comprises producing a target sequence expressed by the first organism. In some embodiments, a target sequence from each organism of the group of second organisms is added to the nucleic acid molecule. In some embodiments, all possible synonymous mutations that produce target sequences from the second organism and do not produce a target sequence from the first organism are produced.

### Origin of Replication Optimization

By another aspect, there is provided a method of for engineering a nucleic acid molecule comprising a coding region, the method comprising extracting sequence features that promote replication from origins of replication (ORI) from the first organism and the second organism, generating an ORI in the nucleic acid molecule that is enriched for sequence features from the first organism and/or depleted of sequence features from the second organism, thereby engineering a nucleic acid molecule.

In some embodiments, the generated ORI is an artificial ORI. In some embodiments, artificial is synthetic. In some embodiments, the generated ORI is a composite ORI. In some embodiments, the artificial ORI is a composite ORI. In some embodiments, a composite ORI comprises a plurality of different ORIs. In some embodiments, a composite ORI comprises features from a plurality of different ORIs. In some embodiments, the generated ORI is enriched for sequence features from the first organism. In some embodiments, the generated ORI is depleted of sequence features from the second organism. In some embodiments, depleted is devoid of.

In some embodiments, generating an ORI comprises performing hierarchical clustering of the extracted features. In some embodiments, the features from the first organism are clustered. In some embodiments, if a distance between clusters is greater than a predetermined threshold all clusters with distances above the threshold are included in the nucleic acid molecule. In some embodiments, a composite ORI comprises all the clusters. In some embodiments, if the distance between clusters is less than the predetermined threshold a single cluster is generated. In some embodiments, the single cluster is the artificial ORI. In some embodiments, the single cluster is related to all ORI sequences in the nucleic acid molecule. In some embodiments, the single cluster is related to all ORI sequences in the nucleic acid molecule comprising all said clusters. In some embodiments, the single cluster is related to all ORI sequences extracted. In some embodiments, if the distance between clusters is less than the predetermined threshold a single artificial ORI is generated comprising a single cluster that is related to all the ORI sequences in the cluster that were below the threshold. A skilled artisan will understand that for sufficiently similar clusters a single artificial ORI can be generated that will encompass all those similar clusters. But when clusters are two dissimilar a compound ORI will be generated that is a merging of the two clusters. In some embodiments, an ORI from each organism of the first set of organisms is included in the composite ORI.

In some embodiments, the method comprises producing at least one mutation in an ORI. In some embodiments, the mutation in made in the artificial ORI. In some embodiments, the mutation produces a sequence feature from the first organism. In some embodiments, the mutation removes a sequence feature of the second organism. In some embodiments, the method comprises selecting at least one feature from at least one cluster from the first organism and including it in the molecule. In some embodiments, the method comprises selecting at least one feature from at least one cluster from each organism of the first set of organisms and including it in the molecule. In some embodiments, the method comprises removing from the molecule at least one feature from at least one cluster from the second organism. In some embodiments, the method comprises removing from the molecule at least one feature from at least one cluster from each organism of the second set of organisms.

### Interfering RNA Generation

By another aspect, there is provided a method of for engineering a nucleic acid molecule comprising a coding region, the method comprising identifying at least one gene expressed in the second organism and introducing into the nucleic acid molecule at least one portion of the at least one identified gene, thereby engineering a nucleic acid molecule.

In some embodiments, the identified gene is highly expressed in the second organism. In some embodiments, the identified gene is exclusively expressed in the second organism. In some embodiments, the identified gene is not highly expressed in the first organism. In some embodiments, the identified gene is not expressed in the first organism. In some embodiments, the identified gene is essential to the second organism. In some embodiments, the identified gene is not essential to the first organism.

In some embodiments, the portion comprises at least 10, 12, 14, 15, 16, 18, 20, 21, 22, 23, or 25 nucleotides. Each possibility represents a separate embodiment of the invention. In some embodiments, the portion is of a size sufficient to act as an interfering RNA. In some embodiments, the portion is between 21 and 23 nucleotides. In some embodiments, the interfering RNA is an siRNA. In some embodiments, the portion comprises at most 23, 25, 30, 35, 40, 45, 50, 60, 70, 80, 90 or 100 nucleotides. Each possibility represents a separate embodiment of the invention. In some embodiments, the portion is about 80 nucleotides. In some embodiments, the interfering RNA is an shRNA. In some embodiments, acting as an interfering RNA is after transcription. In some embodiments, acting as an interfering RNA is after cleavage. In some embodiments, acting as an interfering RNA is after Dicer cleavage.

In some embodiments, the portion is introduced into an open reading frame. In some embodiments, the portion is introduced into a coding region. In some embodiments, the portion is introduced into an exon. In some embodiments, the portion is introduced into an intron. In some embodiments, the portion forms a hairpin. In some embodiments, the portion is flanked by two sequences that form a hairpin. In some embodiments, the portion is flanked by sequences that are targets of Dicer/Drosha.

### Uptake Signal Optimization

By another aspect, there is provided a method of for engineering a nucleic acid molecule comprising a coding region, the method comprising optimizing intergenic sequence in the molecule by enriching with uptake signal sequences (USS) from the first organism and/or depleting USS from the second organism, thereby engineering a nucleic acid molecule.

In some embodiments, the optimizing comprises enriching for USS form the first organism. In some embodiments, the optimizing comprises depleting USS form the second organism. In some embodiments, the enriching is in the intergenic sequence. In some embodiments, the depleting is in the intergenic sequence. In some embodiments, intergenic sequence is intergenic region.

In some embodiments, the optimizing uses the Chimera algorithm. In some embodiments, the algorithm is implemented based on suffix trees. In some embodiments, the optimizing comprises selecting subsequences enriched in the first organism. In some embodiments, the optimizing comprises removing subsequences enriched in the second organism. In some embodiments, a subsequence comprises at least 4, 5, 6, 7, 8, 9, 10, 12, 15, 17, 20, 25, 30, 35, 40, 45, 50, 60, 70, 75, 80, 90 or 100 nucleotides. Each possibility represents a separate embodiment of the invention. In some embodiments, a subsequence comprises at most 10, 12, 14, 15, 17, 18, 20, 25, 30, 35, 40, 45, 50, 60, 70, 75, 80, 90, 100, 150, 200, 250, 300, 400 or 500 nucleotides. Each possibility represents a separate embodiment of the invention.

In some embodiments, the method further comprises outputting an artificial sequence of the engineered nucleic acid molecule.

According to another aspect, there is provided a computer program product comprising a non-transitory computer-readable storage medium having program code embodied thereon, the program code executable by at least one hardware processor to perform a method of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

By another aspect, there is provided an engineered nucleic acid molecule produced by a method of the invention.

By another aspect, there is provided a composition comprises the engineered nucleic acid molecule.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods:

Genes are expressed using a combination of different mechanisms and regulations. The process of evolution is driven by the occurrence of small and random changes, due to the inherent inaccuracy of biological machinery. These changes are then subjected to the power of natural selection, dictating which ones will be passed to future generations. As they drive the process of differentiation into species and phylogenetic progression, they affect every machinery and component in the cell, including the parts related to gene expression.

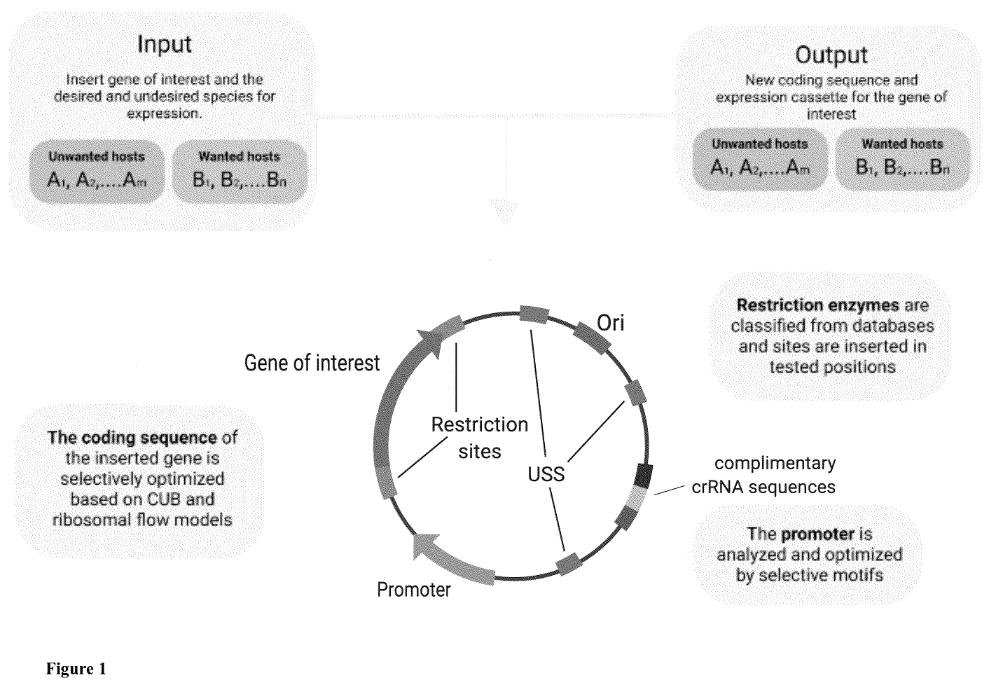

By creating biophysical models for gene expression and regulation processes, the cumulative effect of random alterations is highlighted and emphasized, facilitating the different species-specific preferences of the agents which carry out these processes. Herein, three main steps in this process are focused on, starting from the entry of foreign genetic information into the cell, through the process of transcription up until the translation into proteins (FIG. 1). The synergistic effect of engineering all these genetic elements allows the engineered sequence to be expressed differently in different hosts in terms of efficiency and expression rate, and in terms of the fitness burden they impose on their host.

Fitting genetic elements to a microbiome is defined herein in a rather generic manner. Once the gene itself is selected, there are two sub-communities of interest; first is the community of organisms that should be able to express the modification and will be referred to as the “wanted hosts”. Similarly, the second group is called the “unwanted hosts” since they should have impaired expression of the gene. The goal of the optimization process is to increase expression in the set of wanted hosts, while simultaneously decreasing expression of the same sequence in the unwanted host, considering the fitness effect on both sub communities.

These biophysical algorithms are then used to accumulate and combine the small differences occurring between different species, amplifying their collective effect while remaining within the limitations of noisiness of metagenomic data. The architecture and engineering process facilitated by the algorithms, along with the use of additional databases, helps fill in the missing details and characterization automatically. However, in order to utilize the full potential of the microbiome specific collected data, additional data can be submitted and analyzed flexibly.

The algorithms exhibited in FIG. 1 were specifically designed to leverage the degrees of freedom in the appropriate genetic elements, specifically focusing on those that are easily derived from conventional characterization of the microbiome, meaning through metagenomic techniques. However, they are also designed to accommodate additional information gathered on the community or some of its' species, in order to ensure full utilization of the existing data and the best possible fit to the objective. To sum up, the defined objective and consecutive designing process are essentially used in order to create a maintainable distinguishment between the set of wanted and unwanted hosts, aiming to differentiate expression levels despite constantly occurring horizontal gene transfer (HGT).

Materials and plasmids: PCR master mix, DpnI, Gibson Assembly kit, PCR cleaning kit, competent E. coli and plasmid miniprep kit were purchased from NEB. Agarose for DNA electrophoresis, Chloramphenicol, M9 minimal media and 96-well black plates were purchased from Sigma. LB and agar were purchased from BD Difco, and Ethidium Bromide solution was purchased from Hylabs. Modified versions of gene of interest (GOI) and primers were synthesized by IDT.

Solutions: Bacillus transformation (BT) solution: 80.5 mM dipotassium dihydrate, 38.5 mM potassium dihydrogen phosphate, 3 mM trisodium citrate, 45M ferric ammonium citrate, 2% glucose, 0.1% casein hydrolysate, 0.2% potassium glutamate and 10 mM magnesium sulfate in DDW.

Trace elements solution (×100): 123 mM magnesium chloride hexahydrate, 10 mM calcium chloride, 10 mM iron chloride hexahydrate, 1 mM manganese chloride tetrahydrate, 2.4 mM zinc chloride, 0.5 mM copper chloride dihydrate, 0.5 mM cobalt chloride hexahydrate and 0.5 mM sodium molybdate.
Minimal medium: 1× M9 solution, 1× trace elements solution, 0.1 mM calcium chloride, 1 mM magnesium sulfate, 0.5% glucose, and chloramphenicol (5 μg/ml).

Plasmid construction: software-designed mCherry genes were synthesized by IDT and cloned into AEC804-ECE59-P43-synthRBS-mCherry plasmid, to replace the original mCherry gene via Gibson assembly method. Briefly, the original mCherry gene was excluded from the vector by PCR, with primers containing complementary tails to each of the software-designed mCherry genes. PCR products were treated with DpnI to degrade the remains of the original vector and cleaned with PCR cleaning kit. Next, each software-designed mCherry gene was cloned into the vector by Gibson assembly with 1:2 molar ratio (vector:insert) and transformed into competent E. coli. Positive colonies were confirmed by colony PCR and sequencing, and the new plasmids were extracted with miniprep kit.

Bacterial transformation: all plasmids harboring the modified mCherry genes were separately transformed into competent E. coli k−12 following the standard protocol, and into B. subtilis PY79. For the latter, one bacterial colony was suspended in BT solution (see solutions) and grew at 37° C. for 3.5 hrs. Then, the plasmid was added to the bacterial solution (1 ng/1 ul), and following 3 hrs incubation, bacteria was spread over pre-warmed agar plates.

Fluorescence measurement assay: for each tested mCherry gene, a single colony containing the modified plasmid was grown overnight in LB medium. Then, bacterial suspension was centrifuged and resuspended in PBS×1 twice. Following the second wash, the bacterial suspension was centrifuged again, and the pellet was resuspended in minimal medium (see solutions). The bacterial suspension was allowed to grow for 4 hrs. Then, bacteria were diluted with minimal medium to obtain an OD 600 nm of 0.2, loaded into a 96-well plate and grew for 17 hrs at 37° C. with continuous shaking. Fluorescence (ex/em: 587/610 nm) and bacterial turbidity (at OD 600 nm) were measured every 20 min. Each sample was tested in triplicates at three independent experiments.

Computational log-phase detection: growth curves (OD 600 nm) were plotted over time, and linearity that represents this phase was detected by sequential removal of the last point in the linear phase. Then, a linear trendline was fitted to the curve, and if the removal of the point increased the slope of the curve, that point was considered not part of the log phase. These iterations were conducted continuously until ⅛ of the graph is left or if two iterations did not change the calculated slope.

Statistical analysis: We calculated P-values with a permutation test. Briefly, for every optimization, the three experiments from the same organism were averaged and a difference between E. coli and B. subtilis was calculated. Then, splitting, averaging, and distance calculations were performed, to assess if the separation between E. coli and B. subtilis is significant. The P-value is defined as the percent of splits in which the difference between the two is larger than the difference between the original split.

### Example 1: Translation Efficiency Modeling

The open reading frame (ORF) is the genetic element that codes for amino acids. Due to the redundancy of the genetic code, cellular machinery has adapted to translate certain codons more optimally than others, a bias quantified in calculated Codon Usage Bias (CUB) scores. The proposed cellular effect is that ribosomes are a limited resource in living organisms, and so-called “synonymous” changes in the ORF may influence the ribosomal flow, translation efficiency and fitness and can also affect other gene expression steps. Optimization according to CUB, also referred to as codon harmonization, is traditionally meant to optimize expression for a single organism. This algorithm describes the synonymous recoding of the ORF not for a single organism, but for an entire consortium. During this process, the expression and fitness is optimized for the wanted hosts and deoptimized for the unwanted hosts.

Based on the conclusions reached from various Ribo-seq/RNA-seq experiments and ribosomal flow models, a gene's coding sequence can be recoded in order to optimize and deoptimize translation efficiency simultaneously in different organisms.

Translation initiation: The base pairs before the translation initiation site (TSS) and the first codons following it must ensure efficient initiation of the translation process, and therefore are globally optimized for various features, including but not limited to the Shine-Dalgarno sequence (a site complementary to the rRNA, which promotes the binding of the ribosome to the mRNA and translation initiation), folding energy, slower translation, etc.

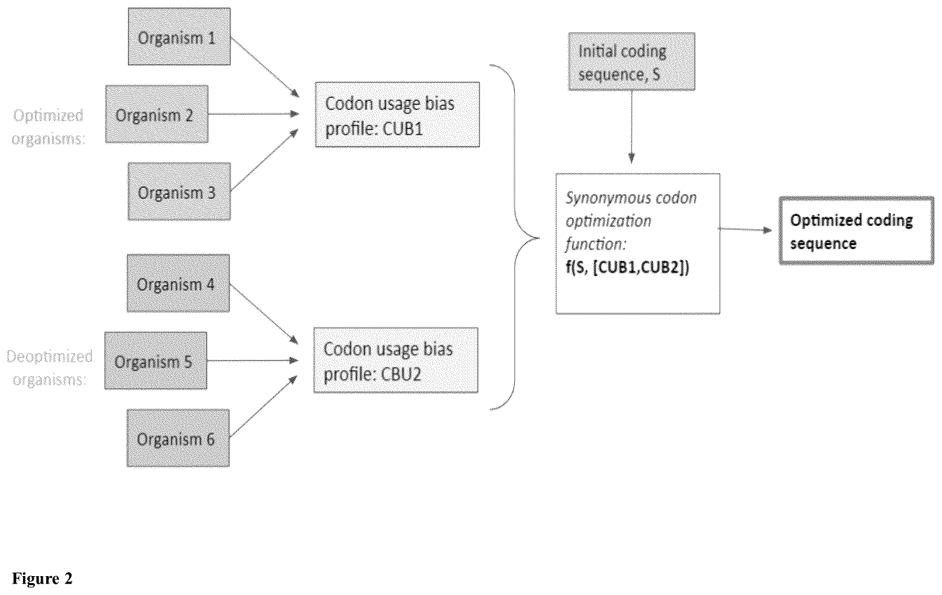

Translation elongation: Changes in translation efficiency of different codons have occurred during species differentiation, creating unique codon usage biases for different organisms. These differences cause a biophysical effect exhibited by the “sliding” movement of the ribosome on the mRNA transcript. Preference of a certain codon over other synonymous options indicates that the ribosome is able to decode it more efficiently, decreasing the burden of translation and thus sliding more easily and freeing up cellular resources. The overall method of translation optimizing is depicted in FIG. 2.

Codon usage bias preferences can be calculated under various assumptions and quantified by different indexes, according to the available data for the microbiome. In this work, we used the Codon Adaptation Index (CAI) which estimates that tendency of an ORF to include optimal/frequent codons and tRNA Adaptation Index (tAI) which measures that adaptation of a coding region to the tRNA pool for characterizing hosts' CUB scores. The Typical Decoding Rate (TDR) can also be used.

Since ribosomes are a limiting resource in cells, these so called “synonymous changes” in the ORF may have a significant in-vivo effect by influencing the flow of ribosomes on the mRNA sequence, thus modulating translation efficiency and overall fitness. Utilization of CUB for gene expression optimization is commonly referred to as “codon harmonization”. In order to create a novel optimization technique based on the traditional one, there are two main challenges to face:
1. Codon harmonization is used in order to increase translation efficiency of a sequence for a specific organism, meaning in the context of a single proteome, considering a single set of gene expression machinery. For the objective of this engineering process, the preferences of the entire microbiome must be taken into account (more specifically, the organisms deemed as relevant for the engineering process).
2. Instead of solely optimizing expression, facilitation of increasing expression for the set of wanted hosts should be coupled to impairment of expression in the unwanted hosts.

The degree of complementarity between the sequence and its' corresponding microbiome should be carefully modulated considering these concerns.

Data for CUB calculation: various techniques and points of view can be used in order to emphasize different aspects of the CUB, usually limited by available data. This tool is equipped to deal with the two main methods to do so; the first looks at the frequencies of different codons in different types of genes, assuming highly expressed genes are likely to have optimal codons, called the codon adaptation index CAI. Secondly, the tRNA adaptation index (tAI) takes into account the supply of different tRNAs and the demand of codon frequencies, considering the interaction strength between the codon and the anticodon. Both options are available and should be used in accordance with the available data regarding the selected microbiome.

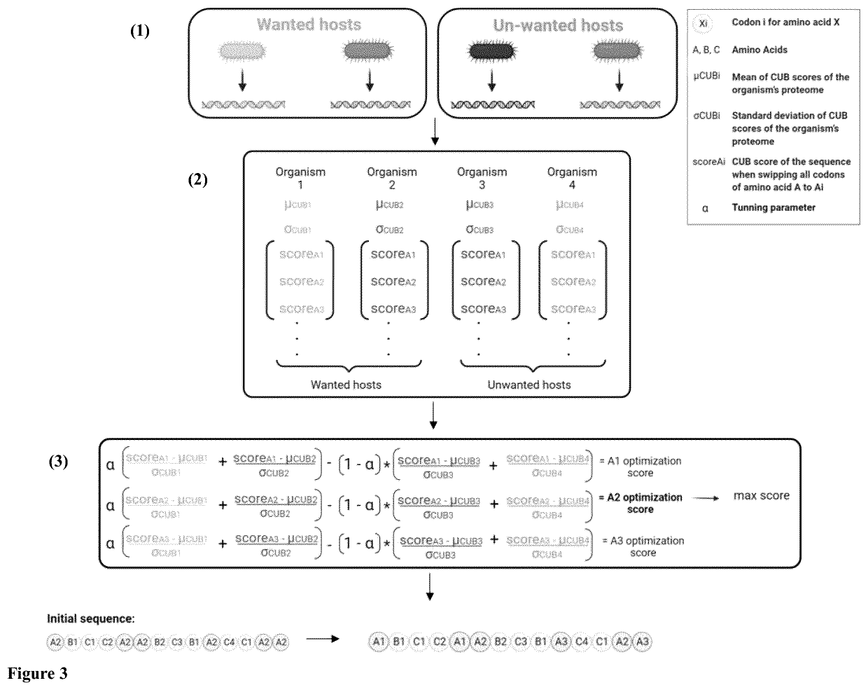

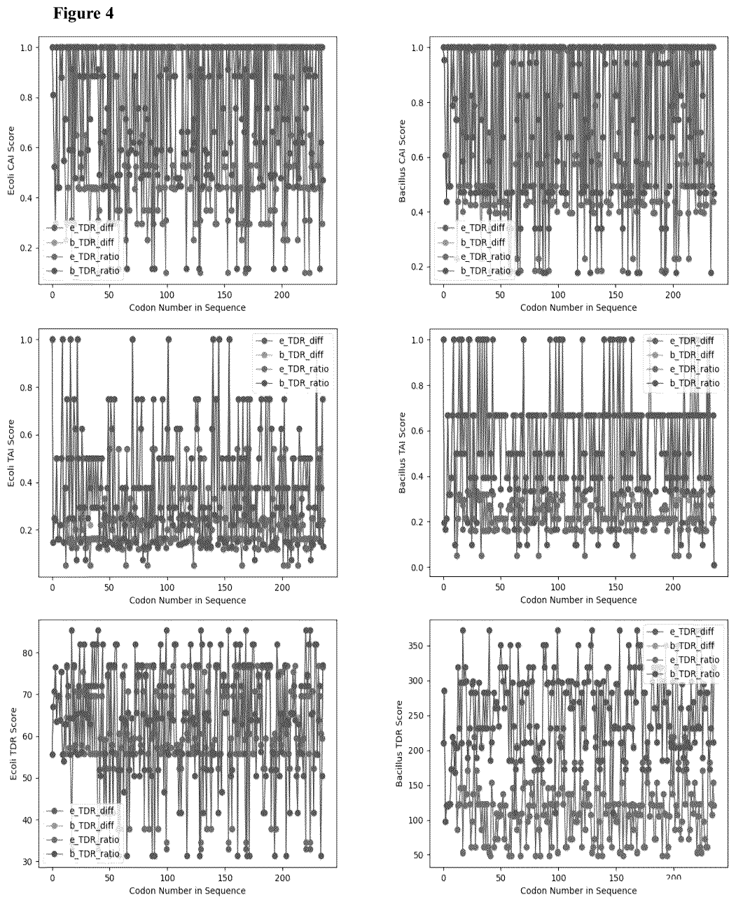

Once the CUB scores of each organism are calculated, the most important features are the mean (μ) and standard deviation (σ), calculated for every considered proteome (meaning all wanted and unwanted hosts (FIG. 3, section 2).

Codon adaptation index (CAI): the underlying assumption is that highly expressed genes have a higher selective pressure to be optimally expressed, thus they are more likely to be consistent of codons that are translated efficiently. In other words, the penalty of having a non-optimal codon out of the synonymous options is much higher in terms of fitness in highly expressed genes compared to lowly expressed genes [19]. According to this understanding, a set of highly expressed genes is obtained and defined as the reference set, either by measuring the protein or mRNA expression levels, or by choosing a set of genes that are known to be highly expressed by homology (such as ribosomal proteins).

Each codon has a usage score wᵢ, named the reference set usage score (RSCU) [19], that is calculated based on a normalized version of the frequency of each synonymous codon xᵢ for amino acid x.

$${w}_{i} = \frac{{RSCU}_{codon}}{{RSCU}_{max}} = \frac{\frac{{x}_{j}}{\hat{x}}}{\frac{{max}_{1 \leq j \leq d} {x}_{j}}{\hat{x}}} = \frac{{x}_{i}}{{max}_{1 \leq j \leq d} {x}_{j}} \qquad ( 1 )$$

In order to translate the scores from single codon scores to scoring a gene with L codons, the geometric mean calculation is applied to all RSCU scores of the codons of a gene of interest (GOI).

$${CAI}_{seq} = {( {\prod }_{i = 1}^{L} {w}_{i} )}^{\frac{1}{L}} = exp ( \frac{1}{L} {\sum }_{i = 1}^{L} ln ( {w}_{i} ) ) \qquad ( 2 )$$

tRNA adaptation index (tAI): CAI is calculated from an evolutionary perspective, highlighting the selective pressure effects on fitness. The tAI measure takes a different approach, aiming to capture the effect of interaction strengths between components of the ribosome, and the supply of said reaction components, highlighting factors related to the physiochemical state of the cell. Each synonymous codon is characterized considering the codon-anticodon noncovalent bond strength, and the corresponding abundance of the recognizing tRNA, as each codon can be recognized by numerous tRNA molecules by wobble interactions.

In order to determine the abundance of different tRNAs, the most optimal measurement would be to measure the expression of the tRNA molecules themselves. However, while this method might work very well for gene expression, tRNA molecules are highly modified RNA sequences and are also very similar to each other, making sequencing outputs inaccurate. The selected measure for this purpose is the tGCN, tRNA genomic copy number of the different tRNAs, using the correlation between the copy number of the molecule and its contribution to the tRNA pool.

The reasoning behind this is similar to the reasoning of the CAI measurements, citing fitness considerations, if a tRNA that is highly used has a gene duplication, it will allow the cells to produce more of that tRNA and reduce the expression burden of the specific gene. The calculation of translation efficiency measure Wᵢ of codon i depends on the interaction strength sᵢⱼ and tGCN of all nᵢ recognizing anticodons:

$${w}_{i} = {\sum }_{j = 1}^{{n}_{i}} ( 1 - {s}_{ij} ) * {tGCN}_{ij} . \qquad ( 3 )$$

Normalization of each score and calculation of the final score of each gene are done in the same manner as in the CAI score:

$${tAI}_{seq} = {( {\prod }_{i = 1}^{L} {w}_{i} )}^{\frac{1}{L}} = exp ( \frac{1}{L} {\sum }_{i = 1}^{L} ln ( {w}_{i} ) ) . \qquad ( 4 )$$

TDR: Typical Decoding Rate: This measurement is based on ribosome profiling data (ribo-seq), which provides a snapshot of mid-translation ribosomal position on the mRNA molecules in a cell during certain conditions.
1. The ribo-seq reads are mapped to the CDS of the proteome.
2. The amount of reads per gene is normalized in order to neutralize bias originated in one codon being present in more highly expressed genes.
3. The normalized number of reads mapped to each codon is collected from all mRNAs mapped, and a histogram is constructed from them.
4. An exponentially modified gaussian distribution (EMG) is fit to the constructed curve, and the gaussian mean is extracted as the score of that codon.

When the codon weights/scores are obtained for all examined organisms (w_{optimize}, w_{deoptimize}), a selective CUB score is calculated for each one of the measurements in one of two strategies:
1. Ratio based selection:

$${w}_{i , ratio} = \frac{{w}_{i , deoptimize}}{{w}_{i , optimize}}$$

2. Difference based selection:

$${w}_{i , diff} = {w}_{i , deoptimize} - {w}_{i , optimize}$$

Every time an amino acid appears in the ORF, the codon coding for it will be “silently” changed to the synonymous codon containing the minimal wᵢ score, in order to ensure better translation efficiency in the optimized organisms compared to the deoptimized organisms.

### Example 2: Optimizations for In-Vitro Evaluation

Optimization is based on choosing the “most optimal” codon between the synonymous codons (which encode the same amino acid). The following CUB measurements were calculated for E. coli and B. subtilis:

CAI (codon adaptation index): as previously explained, in this optimization, the ORF contains optimal codons that were calculated by their relative abundance in a reference set of highly expressed genes of the chosen organism.

tAI (tRNA adaptation index): as previously explained, in this optimization, codons with higher translational efficiency are included in the ORF. It was calculated by considering the intracellular concentration of tRNA molecules and the affinity of each codon-anticodon pairing. This index was calculated based on a reference set of highly expressed genes of the chosen organism.

TDR (typical decoding rate): as previously explained, this optimization is based on ribosome profiling data (Ribo-Seq), which provides a snapshot of a mid-translation ribosomal position on the mRNA molecules in a cell during certain conditions.

Whole microbiome optimization: as mentioned, the main goal of this step is to maintain optimization for multiple organisms while doing so in a selective manner. In order to accomplish those requirements two designing techniques were generated. In the first method the selected model examines the total effect of synonymous changes on the whole microbiome by comparing them to each proteome they will integrate into, named the “proteome relative method”. The second method, called the “individual amino acid method”, calculates and assigns a loss to each codon, without considering the relativity of the scores to the innate score of the microbiome. While the first approach is likely to have a higher degree of complementarity to the microbiome, it creates dependencies between the different amino acids as the protein is considered as a whole, instead of examination of each amino acid individually like in the second method. As such, optimization using the proteome relative method is done using a greedy hill climbing algorithm, which can converge into a local minimum and is more computationally intensive compared to the optimization of individual amino acids.

Proteome-relative method: The effect of a quantitative change in the CUB score of a heterologous gene is relative to the endogenous CUB scores of the proteins in the environment-if the CUB scores of the proteome of a species have a wider distribution and a larger standard deviation, a small change in the CUB of the engineered gene might be less significant.

Due to the number of constraints and considerations, the algorithm was designed to function greedily in an iterative manner. The iterations were constructed to modify the selected DNA sequence (X) as following:
The neighborhood of modified sequences during the iteration are sequences based on X which have all their synonymous codons for a specific amino acid s are changed to a specific codon sᵢ, so that X′=X[s→sᵢ].
For each modified sequence, the CUB score in every tested organism A is calculated. In order to compare the scores of different proteomes, each is “normalized” by quantifying the number of standard deviations that differ the CUB of the GOI from the average score of the said proteome:

$$dist ( a ) = \frac{( {CUB}_{a} ( {X}^{′} ) - {μ}_{a}}{{σ}_{a}} \qquad ( 5 )$$

In order to take both optimization and deoptimization for the wanted (A) and unwanted (B) hosts (respectively) into account, the following optimization score is calculated for every engineered sequence X′ in the neighborhood:

$$f ( {X}^{′} ) = α \cdot {mean}_{a ∈ A} dist ( a ) - {mean}_{b ∈ B} dist ( b ) ) = \frac{{\sum }_{a ∈ A} \frac{( {CUB}_{a} ( {X}^{′} ) - {μ}_{a} )}{{σ}_{a}}}{\vert A \vert} - \frac{{\sum }_{b ∈ B} \frac{( {CUB}_{b} ( {X}^{′} ) - {μ}_{b} )}{{σ}_{b}}}{\vert B \vert} \qquad ( 6 )$$

The sequence with the most significant optimization score is considered as the template for the following iteration. Termination conditions include hitting a (local) maximum or exceeding the defined number of iterations allowed (FIG. 3, section 3).

Individual amino acid method: Along with the CAI and tAI codon usage bias measurements described hereinabove, an additional CUB measurement was added, called typical decoding rate (TDR). TDR utilizes ribo-seq data, which is a type of RNA-seq in which only the parts of sequences that were covered by ribosomes are sequenced, providing a snapshot of the ribosome placement in the cell at a given moment. Based on the ribo-seq the typical decoding rate of each of the codons is estimated.

Two optimization strategies were used to select the codon that will facilitate the highest expression levels for B. subtilis balanced by the lowest possible expression levels for E. coli. The weights wᵢ for each score can be calculated with any CUB measurement.

### Ratio Score (R):

$${R}_{i} = {\sum }_{a ∈ A} \frac{{w}_{i}}{max ( \{ {w}_{i} \} )} - {\sum }_{b ∈ B} \frac{{w}_{i}}{max ( \{ {w}_{i} \} )} \qquad ( 7 )$$

### Difference Score (D):

$${R}_{i} = {\sum }_{a ∈ A} ( 1 - {w}_{i} + max ( \{ {w}_{i} \} ) + {\sum }_{b ∈ B} ( 1 - {w}_{i} + max ( \{ {w}_{i} \} ) \qquad ( 8 )$$

The codon with the highest score will be chosen upon synonymous options to encode for its corresponding amino acids. Abbreviations formatted as “CUB scoring method”-optimization score. CAI results were unique since the ratio and difference optimized sequences were identical.

The minimum of the first sum is achieved when the score of the codon in optimized organisms is close to the maximum value possible. The minimum of the second sum is achieved when the score of the same codon is distant from the maximal value (close to the minimum). So, minimization of the loss function brings an optimal solution from both points of view.

The optimization abbreviation consists of the CUB (tAI, CAI, TDR) type followed by the optimization type (R or D), i.e., tAI-D. Additionally, the reason why CAI is written without the optimization type is due to the fact that by chance, the CAI-R and CAI-D sequences are identical.

Result evaluation: a novel evaluation score is defined as the average distance between the cluster of wanted hosts and the cluster of unwanted hosts for an additional score, comparing the normalized changes between the initial and engineered sequence. The optimization score for each organism is defined as:

$${Score}_{a} = \frac{{CUB}_{a} ( {X}^{′} ) - {CUB}_{a} ( X )}{{σ}_{a}} \qquad ( 9 )$$

A positive optimization score means that the sequence was optimized compared to the non-engineered version, thus for wanted hosts the results should be as positive as possible and for unwanted hosts they should be negative. The formula for the final optimization index:

$$Opt . index = {\sum }_{a ∈ A} \frac{{score}_{a}}{\vert A \vert} - {\sum }_{b ∈ B} \frac{{score}_{b}}{\vert B \vert} = {\sum }_{a ∈ A} \frac{{CUB}_{a} ( {X}^{′} ) - {CUB}_{a} ( X )}{{σ}_{a} \cdot \vert A \vert} - {\sum }_{b ∈ B} \frac{{CUB}_{b} ( {X}^{′} ) - {CUB}_{ab} ( X )}{{σ}_{b} \cdot \vert B \vert} \qquad ( 10 )$$

As shown for the scores themselves, a higher index indicates that translation is more efficient in the wanted hosts compared to the unwanted hosts, while a lower score indicates the opposite.

FIG. 4 shows translation optimization for E. coli and B. subtilis. In each graph the scores of the sequences under the tested selective translation measurement (CAI, tAI, TDR) are shown. The sequences are laid out and scored for each position. In each graph, the green sequence is the sequence optimized for the measurement and the red sequence is deoptimized for the sequence.

### Example 3: Transcription Optimization

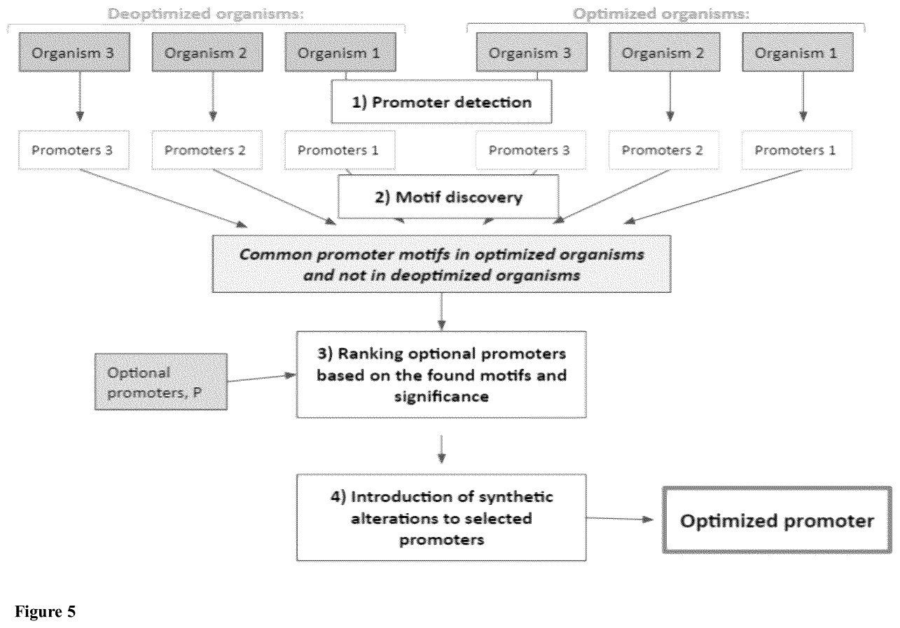

Gene transcription is initialized in prokaryotes by the recognition of promoter sequences, which are found up-stream to a gene, and the recruitment of TFs to allow RNA polymerase to initiate transcription. The core promoters are defined as the exact segment to which the sigma factor in bacterial RNA-polymerase binds. While core promoters are quite universal, upstream regions contain additional sites that are recognized by TFs. Different TFs, utilized by different organisms, recognize different sets of genomic sequences known as “motifs”. By characterizing motifs that are specifically recognized by wanted and unwanted hosts' cellular machinery, the transcription module estimates which promoters will promote transcription initiation only in the group of wanted hosts within a microbiome. These motifs are then used to synthetically design a promoter to enhance expression in one group of organisms and not in the other. The overall method of transcription optimization is depicted in FIG. 5.

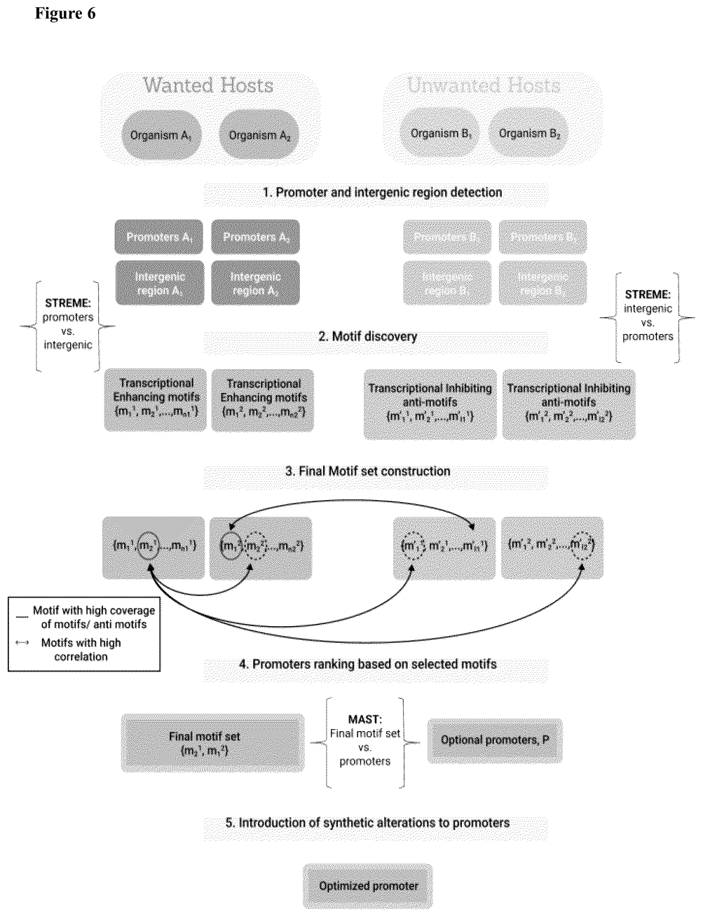

For the purpose of this study, promoter sequences were defined as the first 200 bp upstream to the ORF and intergenic sequences as all sequences on the same strand that neither belong to the ORF nor to the promoter sequences (FIG. 6, section 1). Correspondingly, the model of the invention is designed to detect genetic motifs that uniquely promote transcription initiation in one species (compared to another).

Motif discovery: A Position-Specific Scoring Matrix (PSSM) is usually used to represent sequence motifs, as nucleotides can vary in different positions along the sequence. A PSSM of size 4×L contains the probability of each nucleotide to appear in each position of a motif of length L. PSSM probabilities are calculated assuming motif sites are independent one from another and neglecting insertions or deletions in the motif sequence.

The STREME (Sensitive, Thorough, Rapid, Enriched Motif Elicitation) software tool was used to search for enriched motifs in primary set when compared to a set of control sequences. STREME uses hidden Markov model (HMM) to scan the query sequences for enriched motifs of configured length up to a certain significance threshold. In this study, STREME was run with a configuration of third order HMM, motifs' length of 6-20 bp and a p-value of 0.05. Two sets of enriched motifs related to transcription were searched (FIG. 6, section 2).

Transcription enhancing motifs: to ensure a motif is related to transcription activation in wanted hosts, motifs were searched from the third most highly expressed (inferred from expression data or CUB measurements) promoters of each wanted host with the promoter sequences defined as the primary input and the intergenic sequences as the control. Motifs discovered in this run configuration are enriched in sequences associated with gene expression, which likely indicates their desirable regulatory role.

Transcription inhibiting anti-motifs: to verify a motif will not promote transcription in unwanted hosts, motifs were searched for each unwanted host with the intergenic sequences defined as the primary input and the promoter sequences of the third most highly expressed genes as the control. The discovered motifs, termed “anti-motifs”, are common in sequences that are not associated with gene expression, which likely indicates their transcription suppressing attribute.

For an input of n wanted hosts and m unwanted hosts a total of n+m sets of motifs are created.

Single motif set construction: Let A be the set of wanted hosts and B the set of unwanted hosts. ∀a∈A Sₐ is the set of transcription enhancing motifs for host a and ∀b∈B S_{b} is the set of transcription anti-motifs for host b. A final set F of motifs is constructed according to the following steps (FIG. 5.3):

Calculating motif similarity thresholds: The measurement used to quantitate motif similarity is spearman correlation, which is calculated between a pair of PSSMs. In order to determine the basic amount of similarity between motifs to consider, a different threshold is calculated for each organism. ∀h∈A∪B:
PSSMₕ is a set of 100 random PSSMs with lengths 6-20 bp
∀m∈Sₕ, ∀m′∈PSSMₕ spearman correlationcorr(m, m′) between m and m′ is calculated. Let corrₕ={corr(m, m′)|m∈Sₕ, m′∈PSSMₕ}.
Let P_{X}(corrₕ) be the X-percentile of the spearman correlation values. The motif similarity threshold used for host h is defined as Dₕ=P_{X}(corrₕ). X=95 was set to determine motif similarity threshold for each host.

Defining an initial motif set: The initial motif set C is defined as the union of all transcription-enhancing motifs, C=U_{a∈A} Sₐ.

Calculating motif scores: ∀m∈C two motif scores are calculated:

### Motif Score for a Single Organism:

$${δ}_{h} ( m ) = \{ \begin{matrix}1 & if ∃ m′ ∈ {S}_{h} s . t . corr ( m , m′ ) \geq {D}_{h} \\ 0 & otherwise\end{matrix} \} ∀ h ∈ A ⋃ B \qquad ( 11 )$$

Aggregated motif score, using a tuning parameter a for calibrating the ratio of modification in wanted hosts and unwanted hosts:

$$Score ( m ) = α \cdot {\sum }_{a ∈ A} {δ}_{a} ( m ) + ( 1 - α ) \cdot {\sum }_{b ∈ B} {δ}_{a} ( m ) \qquad ( 12 )$$

Constructing the final motif set: Let Score_{F}={Score(m)|m∈C}. Let P*_{Y}(Score_{F}) be the Y-percentile of the aggregated motif scores. The final motif set F is defined as:

$$F = \{ m \vert m ∈ C , Score ( m ) > {{P}^{*}}_{Y} ( {Score}_{F} ) \} \qquad ( 13 )$$

Y=75 was used to calculate final motif score for each motif (FIG. 6, section 3).

Promoter selection and tailoring: MAST (Motif Alignment and Search Tool) is used to align the final motif set to the top quartile of wanted hosts' promoters in terms of gene expression, when gene expression data is available or estimated based on CUB scores calculated from the hosts' genes. Hosts' promoters are then ranked based on the Expect Value (E-value) of those alignments, considering the initial significance of the motif and the quality of the alignment (FIG. 6, section 4). Top ranked promoters, identified as best candidates to promote transcription initiation exclusively in wanted hosts, are further tailored by individually alternating mismatches between the mapped motifs and the promoters (FIG. 6, section 5).

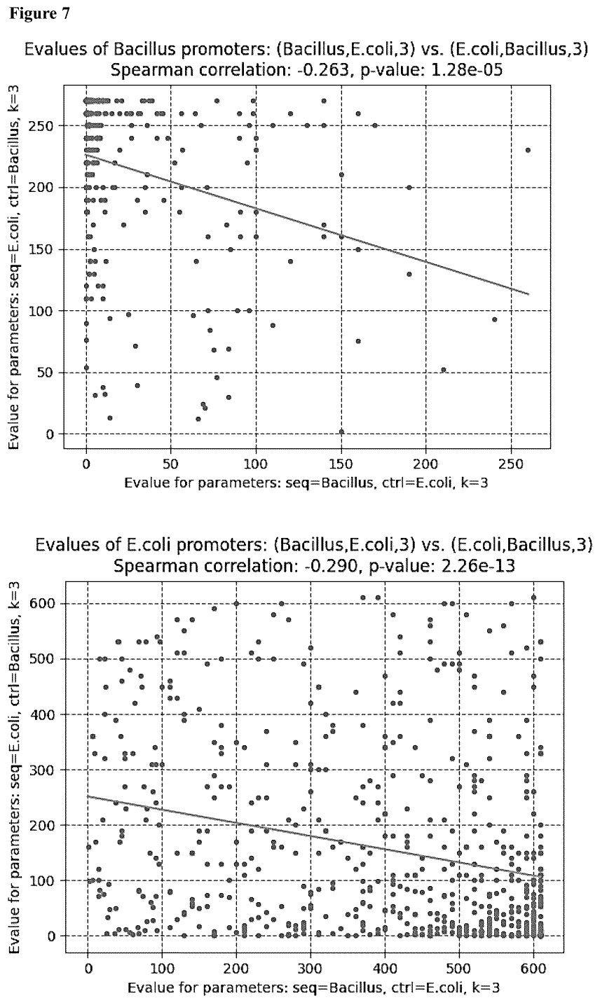

Transcription optimization results for E. coli and B. subtilis are provided in FIG. 7. When finding the motifs of E. coli and B. subtilis in a hidden Markov model (with 3 hidden layers) and examining the correlation between the score each sequence gets given the motifs found in E. coli compared to the score obtained with B. subtilis motifs, it is apparent that there is a significant negative correlation between the two scores. This demonstrates that the motifs are actually able to distinguish between the two organisms.

### Example 4: Editing Restriction Site Presence

Restriction enzymes are the first line of defense in the bacterial immune system, they have the specific ability to recognize a nucleotide sequence and digest it, thus protecting bacteria from the effects of foreign DNA entering it. One of the most variable properties of different bacteria, is their array of recognized restriction sites and footprint of restriction enzymes.

The cleaved product may have different forms, depending on the specific type of restriction enzyme which performed the cleavage action. In some cases, the digestion products have complementary edges that can reattach due to the bacterial DNA repair mechanisms. Therefore, two main factors determine the effectivity of the digestion process: the number recognized restriction sites and the region in which the sites are introduced.

As in other bio-design cases, the topological complexion and scale of effect of the genetic element is directly related to the magnitude of its effect and engineering potential of it. As such, the focus herein is on the ORF and not other genetic elements, in fear of disruption of its action as a byproduct. Changes in the ORF sequence have the most predictable outcome, making them the best candidate for this algorithm.

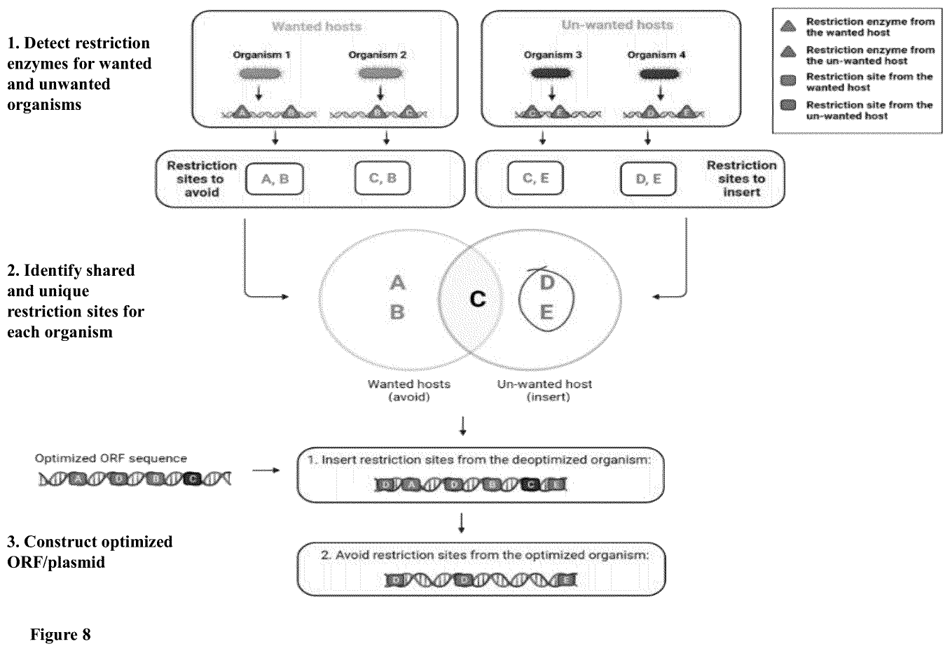

The present invention generates a database of restriction enzymes that are present in the varying organisms. Such data is used first and foremost in order to avoid restriction sites of enzymes that are present in the optimized organisms. Moreover, restriction enzymes that are found only in the deoptimized organisms are examined and corresponding restriction sites are added to various parts of the designed plasmid (the effect of insertion of such sites in different plasmid elements is experimentally tested). This method of the invention is summarized in FIG. 8.

Restriction site detection and filtration: in this preprocessing step, each restriction site is classified as one of the following: sites uniquely recognized by the wanted hosts or unwanted hosts, and sites recognized by both. The goal of this algorithm is to avoid any site present in a wanted host, whether or not it is present in an unwanted host as well, while simultaneously adding sites recognized only by the unwanted hosts without disrupting the sequence of amino acids.

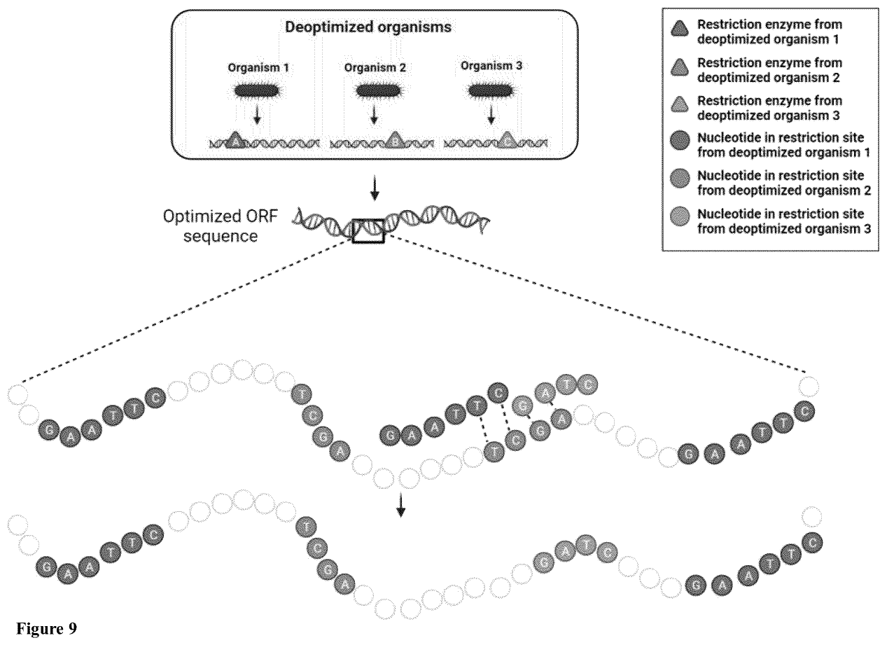

Insertion of sites: overlapping sites can obviously not be inserted together, as the insertion of one site disrupts the presence of the other, thus the objective is to specifically introduce sites that maximize the number of unwanted species that can recognize and digest the sequence, as the total number of present sites is also pursued as a secondary goal. (FIG. 8-9).

In order to increase the overall probability of digestion as defined, all appearances (current and potential-given synonymous changes) are located along the sequence. The first sites to be incorporated are the conflict free sites. In the over-lapping site case, as shown in FIG. 9, sites will be prioritized based on how much they increase the number of unwanted hosts that recognize at least one site in the sequence, and if two options have the same rank, they will be chosen based on the overall number of sites recognized by the unwanted hosts, prioritizing hosts with less found sites.

The order in which the conflicting sites are resolved obviously determine the final DNA sequence to some degree as well, due to the greediness of the algorithm. In order to get the most optimal result given the insertion mechanism, the sites are iterated starting from those with the least complicated conflict up to those with the highest degree of complexity and as result, largest degree of freedom in the final choice of the site.

Avoidance of sites originating from wanted hosts: The sites from the first and third group should be avoided, and their presence in the engineered sequence should be disrupted and altered using synonymous changes, if possible. This algorithm re-writes this requirement as constraints that can be applied to the sequence using the DnaChisel software tool. An important highlight to this method is that the order of these steps is meaningful, as insertion of a restriction site recognized by an unwanted organism can create a new restriction site that might be recognized by a wanted host, reversing the goal of the optimization process.

The Restriction enzyme database (Rebase) is a database of information about two types of enzymes: restriction enzymes, and methyltransferases. The characterization of these enzymes details their origin, recognition sites, and other metadata such as the year of discovery or commercial availability. The detailed sites themselves are noted using standard abbreviations to represent sequence ambiguity, and in some cases note the exact digestion pattern and resulting ends.

The database is constantly updated, as the rate of metagenomic sequencing increases, the fraction of computationally inferred restriction enzymes becomes more prominent (along with reducing rates of biochemical characterization of the sites).

In version 110 (release date: Sep. 28, 2021), there is a total of 4735 organisms (388 of them labeled as ‘Unidentified bacterium’ which were ignored from future analysis), as all strains of the same species were considered altogether. They contain a total number of 5488 sites, with an average of 4.6 sites per organism. The number of sites highly varies, as the standard deviation is 21.2

### Example 5: CRISPR

Similar to restriction enzymes, bacteria use CRISPR as an RNA-mediated defense system which protects against foreign nucleic acids entry into cells, including plasmids. CRISPR (clustered regulatory interspaced short palindromic repeats) is a family of genetic sequences involved in the bacterial/archaeal acquired immune system.

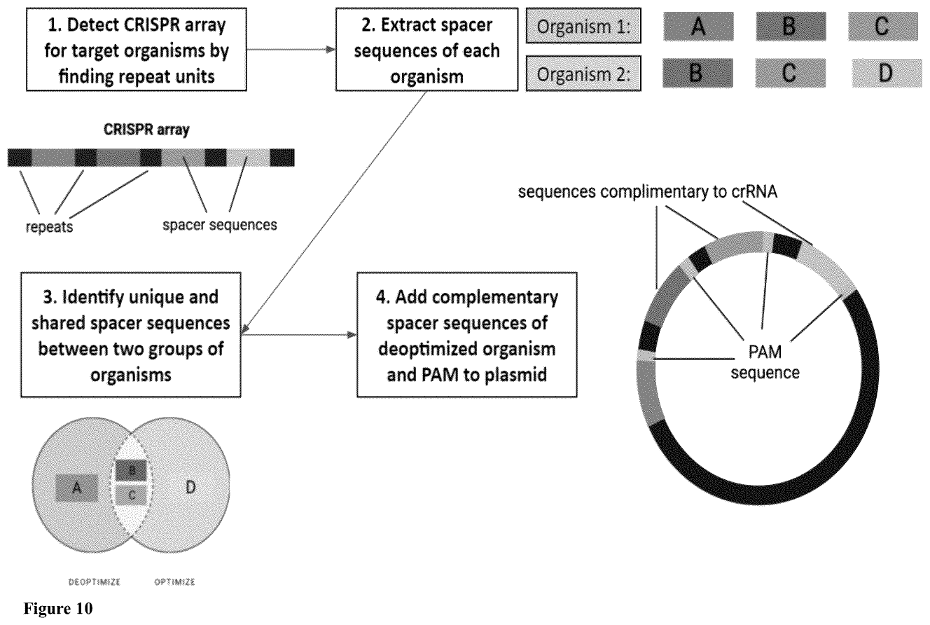

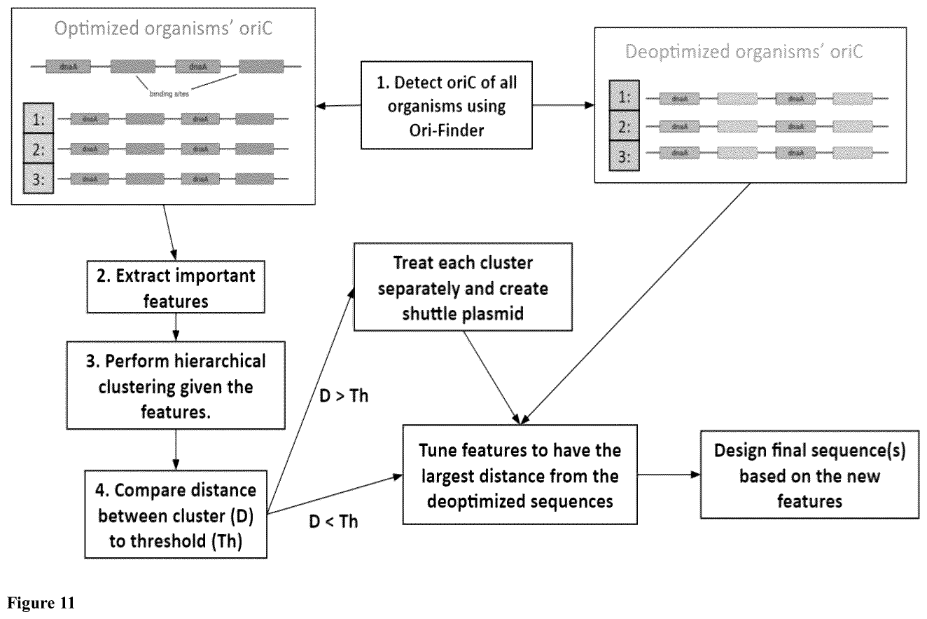

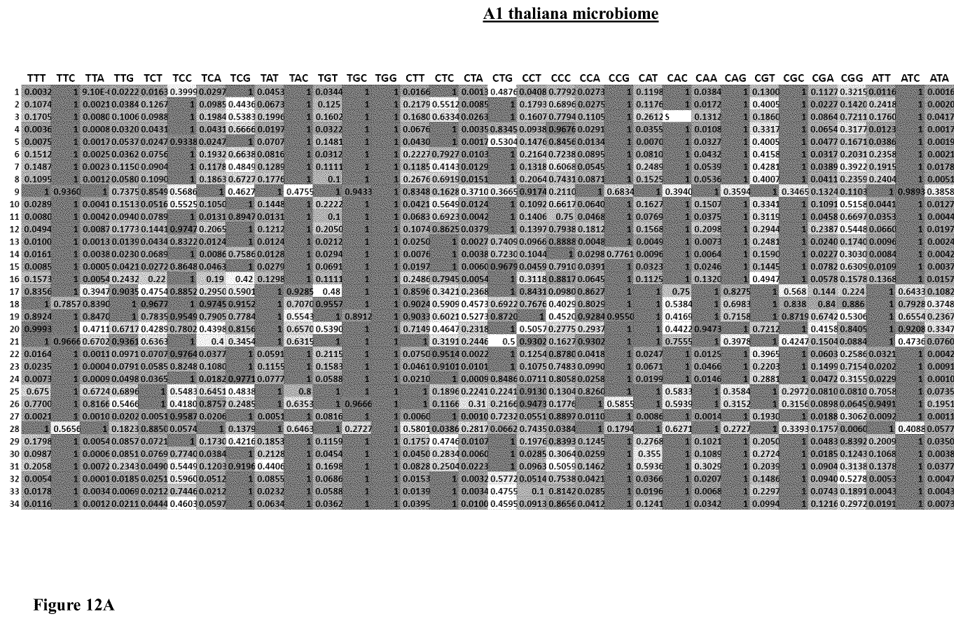

By sequencing the CRISPR system of the different bacteria in the microbial community, the algorithm(s) of the invention identify crRNA (CRISPR RNA) that is uniquely present only in the deoptimized organism. Regions complementary to the specified crRNA are inserted into the designed plasmid along with the corresponding PAM sequence in correct placement (similar to the restriction sites), to promote selective cleavage and digestion of the plasmid in the deoptimized organism. This method of the invention is summarized in FIG. 10.

### Example 6: Origin of Replication

The Origin of Replication (ORI) is the genetic element that promotes replication of the plasmid, it recruits the replication factors to specific binding sites which have highly variable features such as their content, number of occurrences, and the characteristics of the spacer between them. Due to that, the ORI can be carefully tailored to fit the cellular machinery in certain organisms that promotes replication.

The ORI optimization model performs this goal as follows-firstly, it identifies the important features from the ORI genetic elements in both organism groups. Due to the high specificity of the ORI sequence, if two organisms in the optimized group highly differ in their replication machinery, it is best to include a separate ORI for each of them, instead of forcing them into a non-fitting consensus. Thus, the ORI features of the optimized organisms are still analyzed and clustered in the topologically appropriate space, into similar groups, as each group is processed separately.

After the different sequence characteristics are obtained and separated from both organism groups, the sequences of the optimized cluster are compared to those of the deoptimized cluster as the differences between them are tuned and sharpened. Lastly, the different groups are assembled together in order to create a “shuttle plasmid”, which will be able to replicate in the optimized organisms as opposed to the deoptimized organisms. The method of ORI optimization is depicted in FIG. 11.

### Example 7: SiRNA Design

The array of genes which are both unique and highly expressed in a specific organism also has an exploitable variation between species, one which can be used to create RNA probes such as siRNA or gRNA (short interfering RNA and guide RNA correspondingly) in order to achieve directed selection.

For example—if the deoptimized organism contains a unique and highly expressed gene, the gene of interest can be designed to have complementary sites to the defined highly expressed gene, thus causing it to function similarly to a siRNA and repress expression in that organism (and even cause degradation of the mRNA in some cases). Accordingly, the same segment could be inserted into a repressor of the gene in order to promote gene expression in selected organisms.

### Example 8: Transformation (DNA Uptake)

In some bacterial strains, the natural process of DNA uptake is much more efficient when the DNA comes from a homologous sequence. Although homologous sequences do have a higher probability of being recombined into the genome, the biochemical uptake process is selective as well due to the presence of recognized signal sequences named uptake signal sequences (USS). The USS are species-specific consensus sequences distributed randomly between the two strands causing it to be transformable into certain bacterial species.

There are two seemingly opposing explanations for this evolutionary pathway. The “preference first hypothesis”, assumes that the USS is used as a mate recognition signal, as uptake of closely-originated DNA is presumably more beneficial than alien DNA. On the other hand, the “molecular drive hypothesis” supposes that the USS is a result of biased DNA uptake mechanisms.

The USS sequences are distributed randomly between the + and the − strands but tend to appear more in coding sequences than in intergenic regions (and in specific coding frames inside the coding sequences). According to these empirical findings and theories, the model is set to optimize the intergenic sequences present on the plasmid which aren't optimized by any other model, based on the algorithm of the invention. A version of the Chimera algorithm (which is implemented based on suffix trees) can be used to decide if a sequence tends to include many sub-sequences from one group of organisms and less sub-sequences from the second group.

In this model, the bacterial genome for all bacteria is used to calculate a weighted version of the described suffix tree (the last branch in a path is set to have a value equal to the number of occurrences of the corresponding sequence in the bacteria's genome). Afterwards, all the trees belonging to the same group (optimized bacteria, denoted as A or deoptimized bacteria, denoted as B) are combined, as the branches are combined, and their score is set to be the average score between all groups. The two suffix trees are combined together and every “branch” is given a score as a function of the number of occurrences in the optimized organisms and in the deoptimized organismsf(A_occurrences,B_occurrences).

### Example 9: Data Curation for In-Silico Analysis

Evaluation of the results was carried out through both in-silico and in-vitro means, each shedding light on different aspects of the engineering process. The in-silico examination examined the resolution of differentiation between wanted and unwanted hosts, and the sensitivity to the community size and complexity, while the in-vivo experiment was able to quantify the facilitated change in gene expression, and the effect it had on bacterial fitness.

The two main characteristics to be tested for this algorithm are the phylogenetic resolution of optimization, and the ability of this engineering approach to scale up.

The selected microbiome for model analysis is a sample of the A. thaliana soil microbiome, which contained taxonomic lineages and 16S rRNA sequences. The annotated genomes were selected by running the 16S sequence against the BLAST rRNA software (lower threshold for percent identity of the 16S rRNA sequence is 98.5%). As previously mentioned, these algorithms are designed to work with metagenomically assembled genomes in general.

Additionally, the gene used as a target for optimization is the ZorA gene, which serves as a phage resistance gene as part of the Zorya defense system, inferred to be involved with membrane polarization and infected cell death. This gene can be used in a wide array of sub-populations for various different purposes, showcasing the flexibility of this framework.

### Example 10: Translation Efficiency Modeling

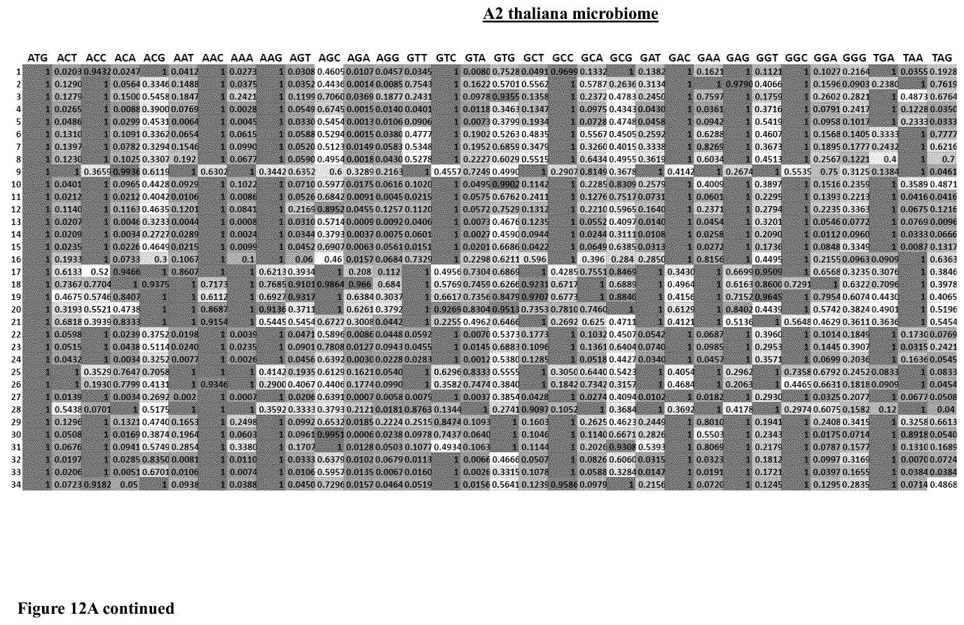

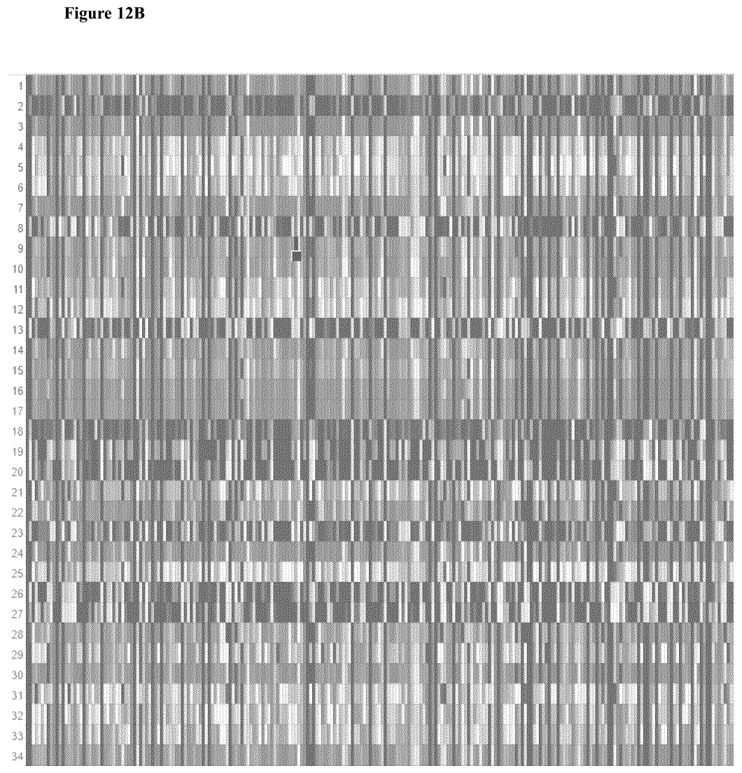

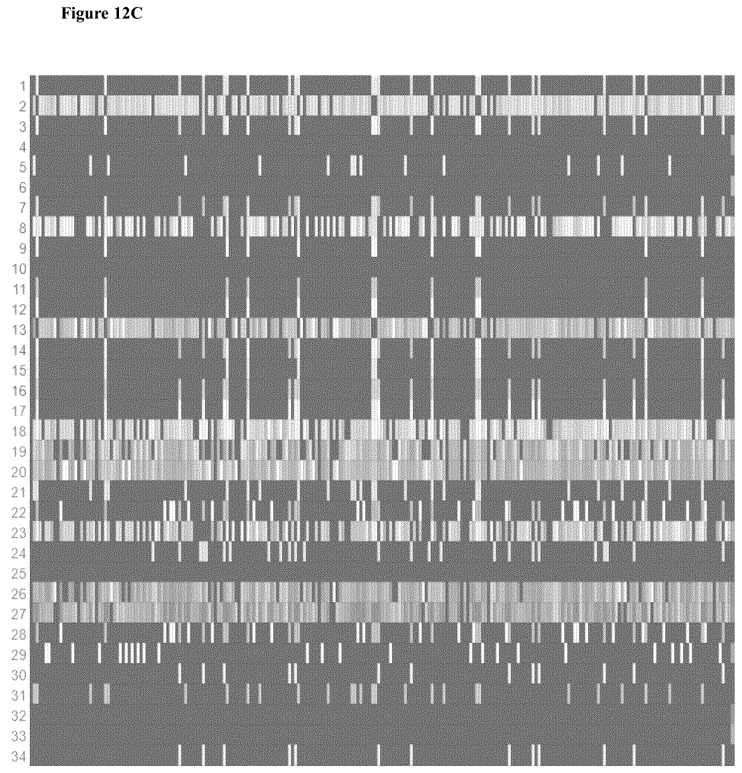

As a start, the effect of an engineering process on the full microbiome was generated and examined. FIG. 12A exhibits the optimization starting point, showing CUB scores of each codon in two examined microbiomes. The organisms found in the microbiomes are listed in Table 2. As expected, the initial scores are relatively diverse, showcasing the potential of modulation of this aspect of gene expression. For a particularly tested gene, FIG. 12B shows the scores of the native sequence and FIG. 12C the scores of the engineered one. Overall, the CUB scores of the optimized sequence are generally regarded to be better compared to the non-engineered version, although the optimization is more substantial for the organisms defined as wanted hosts (organisms 1-16) compared to the unwanted hosts (organisms 17-34).

TABLE 2
Organism in A. thaliana soil

| # in | # in | | Wanted (W) or |
|---|---|---|---|
| FIG. | FIGS. | | Unwant- |
| 12A | 12B-C | Organism | ed(UW) |
| 1 | 22 | Agromyces allii | UW |
| 2 | 29 | Arthrobacter luteolus | UW |
| 3 | 25 | Arthrobacter pascens | UW |
| 4 | 34 | Janibacter limosus | UW |
| 5 | 14 | Knoellia subterranea | W |
| 6 | 33 | Arthrobacter subterraneus | UW |
| 7 | 4 | Arthrobacter crystallopoietes | UW |
| 8 | 6 | Arthrobacter tumbae | UW |
| 9 | 20 | Brevibacterium frigoritolerans | UW |
| 10 | 11 | Mycolicibacterium smegmatis | W |
| 11 | 10 | Nocardioides daejeonensis | W |
| 12 | 12 | Nocardioides jensenii | W |
| 13 | 1 | Nocardioides oleivorans | W |
| 14 | 24 | Nocardioides sediminis | UW |
| 15 | 17 | Nocardioides terrigena | UW |
| 16 | 32 | Paenarthrobacter nitroguajacolicus | UW |
| 17 | 23 | Paenibacillus aceris | UW |
| 18 | 2 | Paenibacillus alginolyticus | W |
| 19 | 18 | Paenibacillus oryzisoli | UW |
| 20 | 8 | Paenibacillus prosopidis | W |
| 21 | 13 | Paenibacillus qinlingensis | W |
| 22 | 9 | Pedococcus badiiscoriae | W |
| 23 | 15 | Pedococcus bigeumensis | W |
| 24 | 30 | Pedococcus dokdonensis | UW |
| 25 | 19 | Peribacillus muralis | UW |
| 26 | 26 | Peribacillus simplex | UW |
| 27 | 3 | Phycicoccus duodecadis | W |
| 28 | 27 | Priestia flexa | UW |
| 29 | 5 | Pseudarthrobacter phenanthrenivorans | W |
| 30 | 21 | Rhodanobacter denitrificans | UW |
| 31 | 3 | Rhodanobacter fulvus | W |
| 32 | 16 | Terrabacter aerolatus | W |
| 33 | 7 | Terrabacter tumescens | W |
| 34 | 28 | Yonghaparkia alkaliphila | UW |

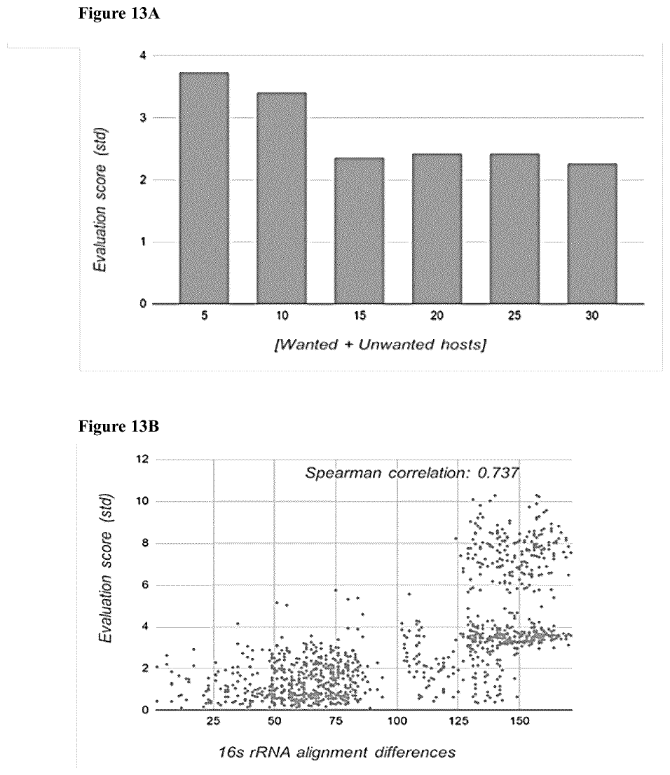

Scale up is tested in FIG. 13A, where the optimization index is calculated for microbiomes of different sizes, and the most evident result is that for all examined sizes the optimization remains relatively similar and significant, roughly 2 units of the defined “standard deviation” compared to the initial sequence. Since the degree of optimization is very loosely dependent on the microbiome size, this phenomenon will be true for larger and more complex microbiomes as well, and they will receive a positive optimization index.

Moreover, the resolution of selectivity that can be achieved by this engineering method, was tested in FIG. 13B. For the analysis, every pair of species in the A. thaliana soil microbiome were selected and subjected to two examinations—the first included calculation of a phylogenetic distance estimate, based on the distance in the 16S sequence alignment between the two species. Next, the algorithm was applied to them (defining one species as wanted and the other as unwanted, reducing the two sets to the size of 1 in order to investigate the direct effects of this factor). The clear correlation (0.737 spearman correlation) between the optimization index and estimated phylogenetic distance sided with expectations given the underlying assumptions on which the algorithm was constructed.

In order to probe the resolution question a bit deeper, the 10% phylogenetically closest pairs were further examined. The mean optimization index is 1.215±0.8, which is a relatively significant optimization in respect to the low degree of phylogenetic diversity presented. In conclusion, these results indicated that this optimization is able to effect realistic sizes and microbiome complexities.

### Example 11: Transcription Optimization

As previously mentioned, promoters have a complex topology, thus the characterization of the effect of any engineering process is less complete compared to other engineered elements. This was taken into account both in transcription algorithm design and analysis, using light selection and modulation in a less direct approach and trying to conserve the innate promoters' structure as much as possible.

The evaluation of the designed algorithm was done in two steps; first the ability to differentiate motifs between wanted and unwanted hosts was closely inspected, and only then was the scale up of the algorithm investigated in a similar manner to the translation efficiency model.

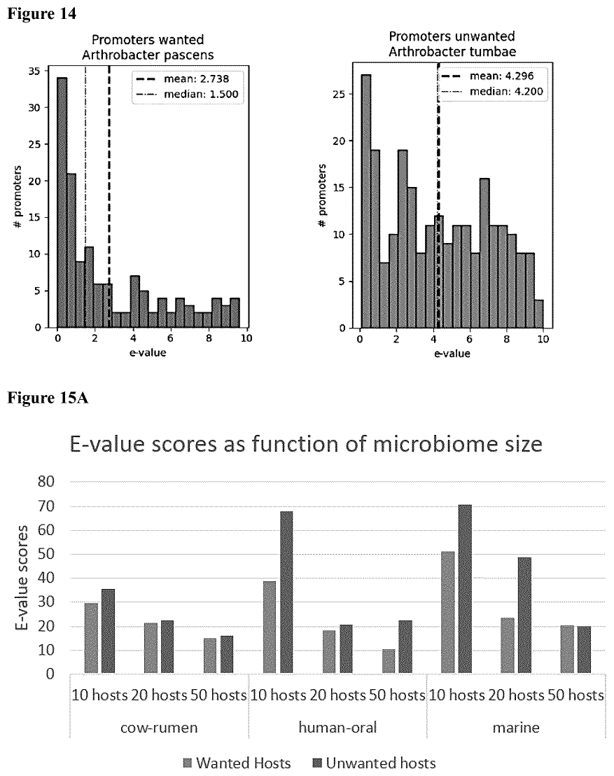

Examination of the differentiation capacity of the algorithm between wanted and unwanted hosts (FIG. 14) was done by examining the ability of the final curated motif set to contrast promoters for two species originating from the A. thaliana microbiome.

Although the two species in FIG. 14 originate from the same phylogenetic family, the E-values of the highly expressed endogenous promoters from the wanted host have better match scores with motifs in the final motif set, compared to the unwanted host. This effect can be seen by two different trends-first, the mean and median of the E-values scores of the highly expressed promoters are lower for the wanted host. Second, the exponential trend in the E-values of the wanted host shows the ability of the algorithm to find a selected set of candidates that can serve as selective promoters. This evidence supports the approach implemented in the module of the invention, that motifs can indeed be used to construct promoter sequences that promote transcription only in a group of wanted hosts within a microbiome.

The dataset chosen for examination of the scale up of the algorithm was the MGnify genome dataset, which has sets of high quality metagenomically assembled genomes (MAGs) for various environments.

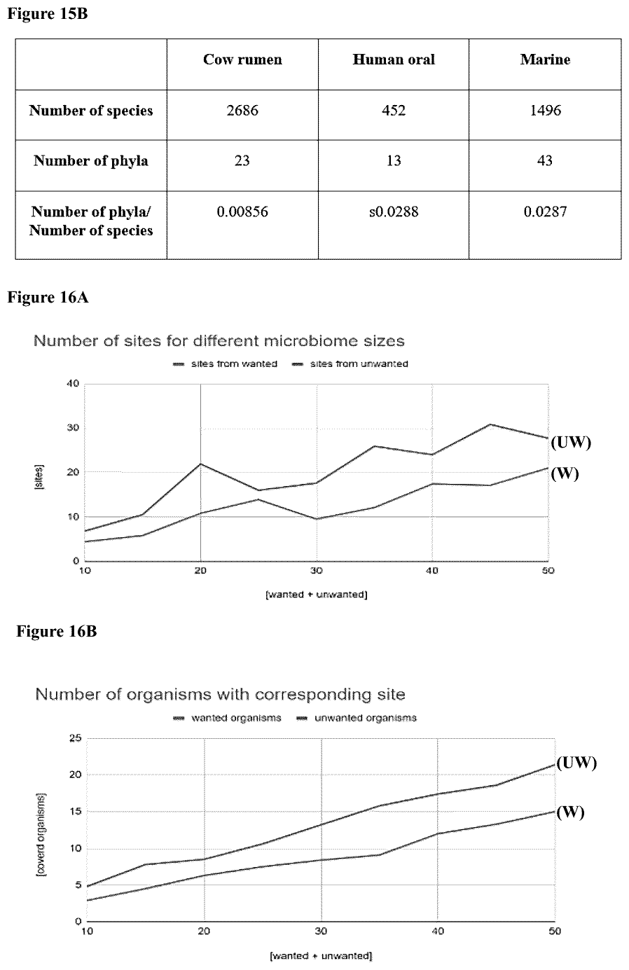

FIG. 15A demonstrates the performance of the transcription module for three different microbiomes from MGnify—the human oral microbiome, the cow rumen microbiome, and the marine microbiome. Each of the Mgnify sets is built using numerous metagenomic projects and contains high quality MAGs. These MAGs were randomly sampled in order to examine the effect of the algorithm on small, medium and large microbiome sizes. The phylogenetic richness and quality of the genomes in the samples were not controlled, mimicking the intended usage of the tool in microbiome research.

The overall trend observed in all three microbiomes is the decrease in E-values scores for increasing microbiome sizes. This result is rather counter-intuitive, as larger microbiomes are more complex and thus are expected to be more difficult to optimize. However, since the algorithm uses a single motif set to fit all wanted hosts, in case of a large phylogenetic variance in the wanted hosts group, as may be the case when using randomized samples of species from different phyla, the performance of the algorithm per each host in the group is sub-optimal. When using larger microbiomes, since the dataset used contains species from a finite set of phyla, it is more likely to select wanted hosts with a closer phylogenetic distance, thus improving the fitness of the motifs in the final set for a larger number of wanted hosts.

Additionally, it should be noted that the cow rumen microbiome has overall lower E-value scores for both wanted and unwanted hosts in comparison with the human oral and marine microbiomes, with less differentiation between wanted and unwanted groups. In terms of the number of species (FIG. 15B), the human oral microbiome has 452 MAGs, the marine microbiome has 1465 and the cow rumen microbiome has 2686. When observing the number of phyla in each microbiome, the ratio between the number of species (represented as the number of MAGs) and the microbiome size seems to be similar and much larger for the human oral and marine microbiomes compared to the cow rumen microbiome. This observation may indicate that the microbiome richness is the key factor influencing the mentioned difference.

In a rich microbiome that has phylogenetically distant species, such as the human oral and marine microbiomes, small sub-microbiomes are likely to have a higher degree of distinction between wanted and unwanted hosts since the overlap between the groups is relatively small. When the size of the sub-microbiome increases, it is more likely to include wanted and unwanted hosts from overlapping phyla, thus minimizing the differentiation between the groups. When examining microbiomes that are less diverse, such as the cow rumen microbiome, randomly selected species of wanted and unwanted groups are likely to be more similar even for small sub-microbiomes, thus reducing the observed effect of microbiome size, as increasing the sub-microbiome size does not incur a proportional increase in the phylogenetic diversity of the wanted and unwanted hosts which isn't already captured for smaller sub-microbiomes.

In conclusion, the analysis exhibits the ability of the transcription optimization model to differentiate between the group of wanted and unwanted hosts.

### Example 12: Editing Restriction Site Presence

In order to capture the full effect of the diversity of known sites and species, the characterized species were used as a pool to select sub-microbiomes, and assess the scale up of the model along with other properties. The optimized sequence is the same one used for ORF optimization of the ZorA phage resistance gene.

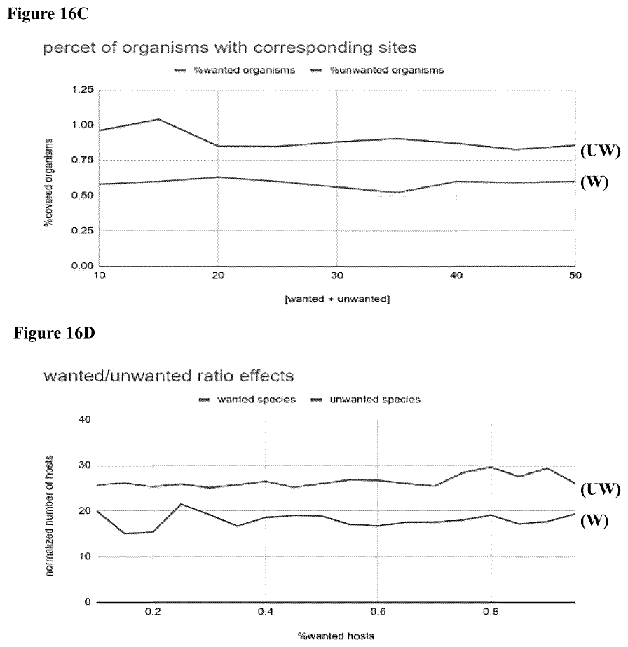

To examine the performance of the algorithm for different sizes of microbiomes, 10 random microbiomes of the tested sizes were optimized and evaluated. After applying the model to the defined microbiome, the number of sites incorporated in the final sequence from each one of the two groups (FIG. 16A), the number of organisms that have a corresponding site (FIG. 16B), and the percent of organisms that have a corresponding site (FIG. 16C) were calculated. Restriction sites recognized by the wanted and unwanted hosts were also normalized (FIG. 16D). For each ratio, the number of species that have a site recognized by a restriction enzyme was calculated for both groups and divided by the total number of species in the group for the sake of normalization. 30 species were randomly chosen and split into wanted and unwanted hosts according to the presented ratio.

An important point to be made prior to more in depth explanations, is to clarify the reason why the engineered sequence even has restriction sites recognized by enzymes present in the wanted group of hosts. A natural assumption could be that this is a byproduct of the insertion of sites from unwanted species, however this is not the case—the last optimization process aims to eliminate the part of this effect that occurs due to that reason. The problem is, restriction sites are innately ambiguous, so in many cases these degrees of freedom are larger than those posed by the redundancy of codons, thus synonymous codon swaps cannot remove the presence of these restriction sites.

In all cases the new version has a larger amount of restriction sites recognized by unwanted hosts compared to wanted hosts, and same goes for the number of species with a corresponding site. These two observations are the main desired outcomes for the algorithm-showing that both a larger number of unwanted species will be able to degrade the engineered sequence, and also the probability of full degradation (associated with the number of recognized sites) is larger in the unwanted hosts as well.

FIG. 16C gives a spotlight to evaluate the ability of the optimization process to scale up to larger microbiomes, by checking the percent of organisms from each group that have a corresponding site in the engineered sequence for all microbiome sizes. The most evident detail is the lack of a specific trend for both groups; 60% of wanted bacteria have at least one restriction site in the engineered sequence, compared to 90% of the unwanted hosts, for all sizes.

And additional interesting property was the effect of different ratios of wanted and unwanted species within the microbiome. Analysis of the effect of this characteristic was done by selecting random microbiomes of 30 species, assigning a certain percentile of them as wanted (as the others are assigned as unwanted species), for percentiles ranging from 5%-95%.

Continuing the trend presented in the scale-up examination, for all tested ratios the algorithm was able to create a degree of differentiation between the percent of the group that has a restriction site between the wanted and the unwanted host, remaining largely indifferent to the presented condition. The main conclusion is that not only is the algorithm able to create a degree of distinction between the wanted and unwanted groups of hosts, but that it is able to do so in a way that fully expresses and accommodated the specific customizations and requirements of the engineering process.

### Example 13: In Vitro Results

To validate the software's ability to design selectively expressed genes, a fluorescence assay was established to monitor the expression levels of the reporter gene mCherry, in two distinct bacteria-E. coli and B. subtilis. The software generated five variants of the gene's ORF that were predicted to be preferentially express in B. subtilis (wanted bacteria), but not in E. coli (unwanted bacteria). Each version was cloned into a plasmid, which was then transformed into both bacteria. The bacteria were allowed to grow separately for 17 hours in a 96-well plate, while the fluorescence intensity that reflected the expression level of mCherry, and bacterial density, were measured for each modified version of mCherry gene (see Materials and Methods). Alterations in these parameters were compared to the unmodified variant of the mCherry gene.

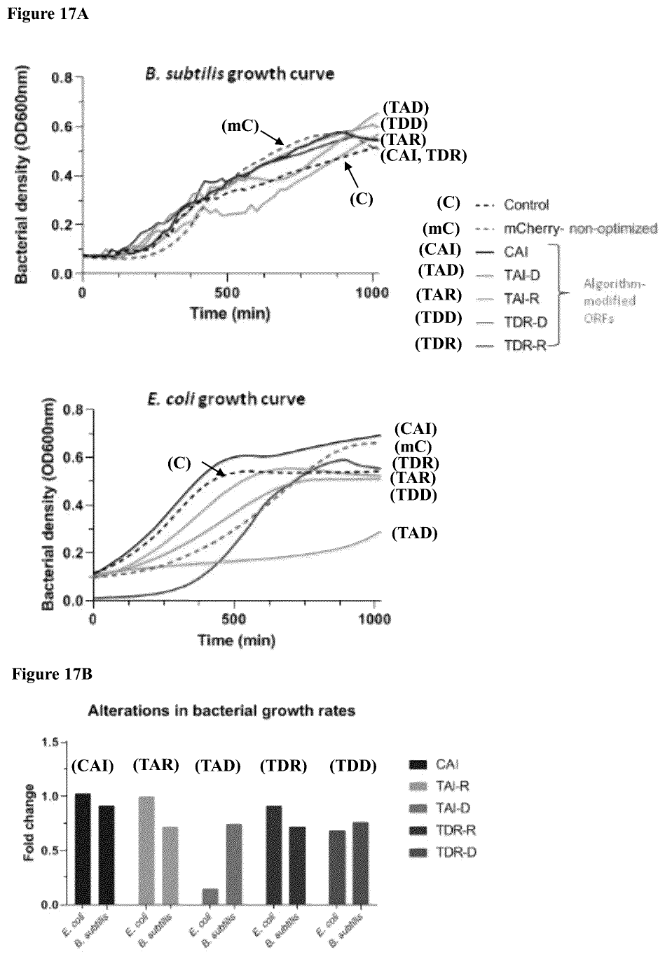

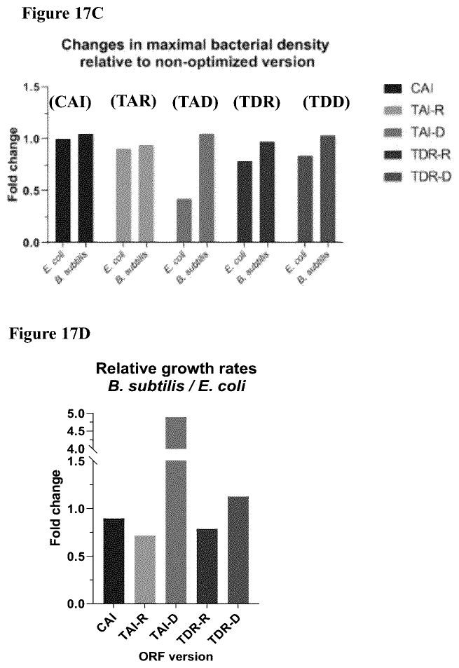

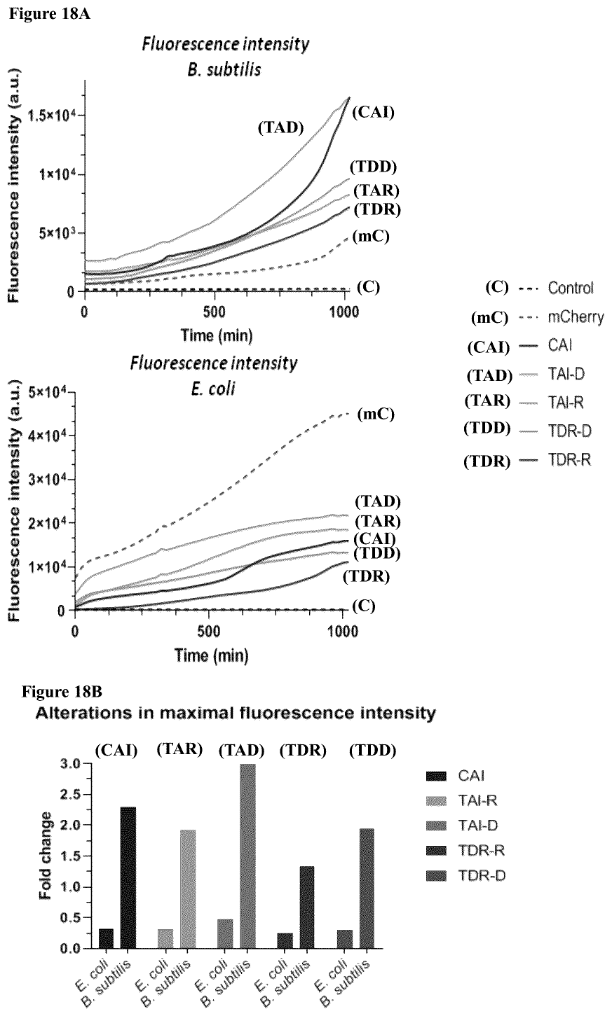

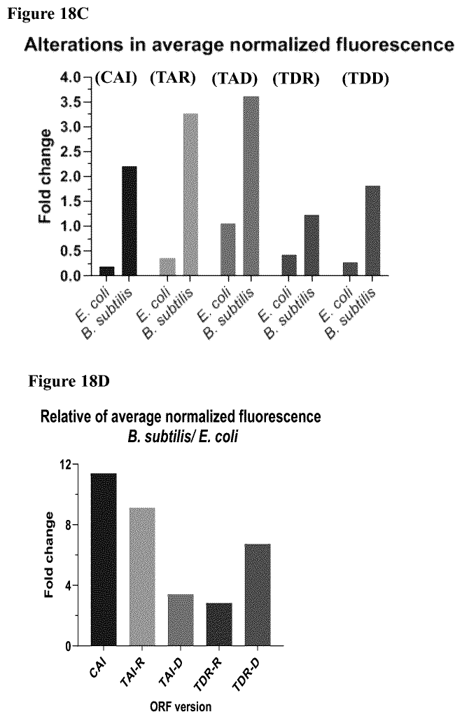

Modifying the gene of interest's ORF arrests the propagation of the deoptimized host: At first, the effect of each mCherry variant on bacterial growth was determined. The measured bacterial density (solution turbidity at 600 nm) was plotted against time (FIG. 17A), and logarithmic phase was detected as described hereinabove. Bacterial growth rates were depicted as the slopes of the logarithmic trendline. In B. subtilis, all modified variants of mCherry exhibited decreased growth rates compared to ‘wild-type’ mCherry, but maximal bacterial densities were similar (FIG. 17B-C). In E. coli, however, variants TDR-D, and particularly tAI-D, showed limited growth rates (up to seven-fold change in tAI-D, FIG. 18B), as well as reduced maximal bacterial density (FIG. 18C). This might be due to ribosomal traffic jams that in turn attenuated overall protein synthesis, and thus restricted bacterial propagation. In order to evaluate the extent of gene selectivity toward B. subtilis relative to E. coli, growth rates folds (modified mCherry version/unmodified mCherry) of B. subtilis were divided by those of E. coli. The mCherry variants TDR-D, and more robustly tAI-D, clearly demonstrated selectivity toward B. subtilis, with regard to growth rates (FIG. 17D).

### Example 14: Expression Levels of the GOI Confirm Model Performance

Examination of mCherry fluorescence intensity, which corresponds to its expression levels in the bacteria, showed that expression of the gene variants correlated with the model predictions. The expression levels over time of all variants were largely amplified in B. subtilis, while substantially diminished in E. coli (FIG. 18A) in comparison to the basal fluorescence of the original variant. These results fit to the chosen optimized and deoptimized bacteria, respectively. It was also evidenced by the average maximal fluorescence intensity for all variants, with dominant effect of tAI-D in B. subtilis that exhibited three-fold increased and TDR-D in E. coli that demonstrated almost four-fold decreased (FIG. 18B). To pinpoint the direct effect of the software gene code modifications on mCherry expression, the expression levels were normalized by bacterial density, by calculating the ratio of fluorescence intensity per bacterial density. Then, average normalized fluorescence and its fold change relative to the original mCherry variant were determined (FIG. 18C). Following normalization, variant tAI-D ranked as the most effective modification in elevating mCherry expression in B. subtilis, while variant CAI reduced mCherry expression to a minimum in E. coli. Intriguingly, normalized fluorescence of variant tAI-D in E. coli was comparable to the unmodified mCherry variant, but also impeded bacterial propagation (FIG. 18D). This implies that gene modification solely based on tRNA abundance might not always be sufficient for expression selectivity, but it eventually halts bacterial growth.

### Example 15: Testing Horizontal Gene Transfer within a Bacterial Consortium

Following initial findings and algorithm adjustments, selectivity of GOI expression is evaluated while horizontal gene transfer (HGT) events occur in a co-culture. For this purpose, chosen bacteria are grown together within the Chi.Bio reactor system for an extended period, to allow horizontal gene transfer to occur. Chi.Bio reactor is a programmable robotic system allowing coculturing and measuring of bacterial density (OD) and fluorescence intensity, without intervention except automatic medium supply and waste removal.

Two sets of experiments are done separately to monitor HGT in a binary bacterial culture, known to exchange plasmid between them (“HGT pairs”), within the Chi.Bio reactor:
1. Fluorescence based measurement-Bacteria A harbors a plasmid containing GOI coding for a fluorescence protein, which is deoptimized for expression in bacteria A, but optimized for bacteria B. If HGT takes place, bacteria B fluorescence levels are detected by Chi.Bio. The system is programmed to maintain logarithmic bacterial growth with occasional fluorescence measurement.
2. Single-cell fusion PCR based detection—This method is also applied on bacterial samples from the fluorescence-based measurement. However, since the possibility exists that the plasmid in bacteria A will lose its stability over time (due to deoptimization and its metabolic burden), which may impair HGT, the experiment is also conducted in the opposite way. Thus, the plasmid is optimized for bacteria A, but deoptimized for bacteria B. Or the plasmid can be optimized for both. In these cases, measuring HGT to bacteria B is quantified by a single-cell fusion PCR as described in Diebold et al., 2021, “Linking plasmid-based beta-lactamases to their bacterial hosts using single-cell fusion PCR”, Elife, July 20; 10:366834, herein incorporated by reference in its entirety. This method enables tracking plasmid distribution and GOI expression among specific community members.

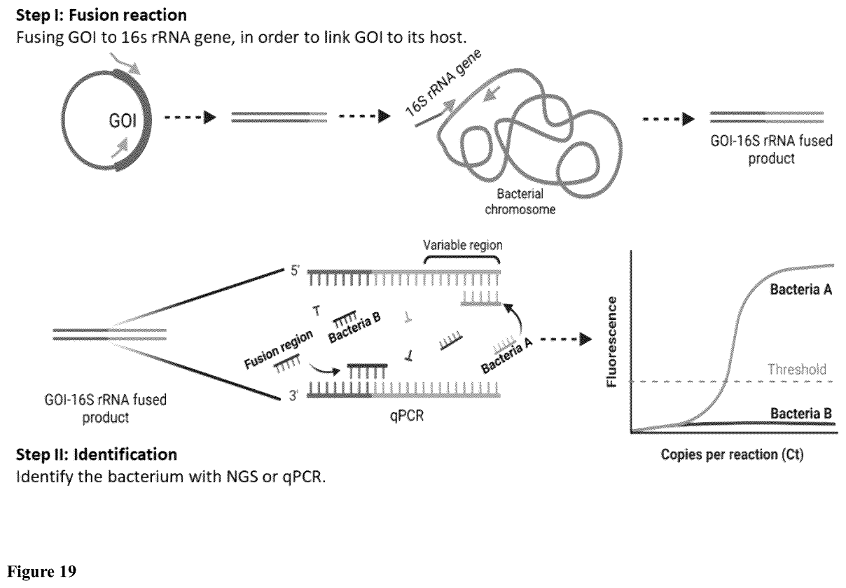

The single-cell fusion PCR method is implemented as follows (FIG. 19): Bacterial community samples at selected time points are emulsified to encapsulate a single bacterium in emulsion droplets. Then, fusion PCR reaction is performed using forward and reverse primers targeting GOI, with a tail attached to the reversed primer targeting V4 region of 16S rRNA gene of each bacterium. Then, the GOI amplicon serves as a forward primer to amplify the V4 region of 16S rRNA gene together with the respective reverse primer. The fused product (GOI-16S rRNA) is cleaned and subjected to qPCR with a specific set of primers targeting the fusion region, in order to assess the incorporation levels of the plasmid in the bacteria.

There is observed an increased qPCR signal when using bacteria B specific 16S rRNA primers, over time, due to HGT from bacteria A that carry the plasmid. This method is also applied to determine expression levels of GOI by using primers for mRNA instead and conducting RT-fusion PCR.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A computerized method for engineering a nucleic acid molecule comprising a coding region optimized for expression of said coding region in a first set of organisms and deoptimized for expression of said coding region in a second set of organisms,
the method comprising at least one of:
a. calculating a codon usage bias (CUB) of said first set of organisms, and a CUB of said second set of organisms and replacing at least one codon of a nucleotide sequence of said coding region with a synonymous codon, wherein said synonymous codon is selected for in said first set of organisms based on said calculated CUB and deselected for in said second set of organisms based on said calculated CUB;
b. receiving a first list of sequences of regulatory elements of highly expressed genes in said first set of organisms and a second list of sequences of regulatory elements of highly expressed genes in said second set of organisms, selecting sequence motifs enriched in said first list and depleted in said second list, engineering an artificial regulatory element comprising a plurality of said selected sequence motifs and operably linking said artificial regulatory element to said coding region in said nucleic acid molecule;
c. determining target sequences of DNA cleaving agents expressed only by said first set of organisms and target sequences of DNA cleaving agents expressed only by said second set of organisms and altering a sequence of said nucleic acid molecule to include at least one of said target sequences of DNA cleaving agents expressed only by said second set of organisms or to remove at least one target sequence of DNA cleaving agents expressed only by said first set of organisms;
d. extracting sequence features that promote replication from origins of replication (ORI) from said first set of organisms and said second set of organisms, generating an artificial ORI in said nucleic acid molecule that is enriched for sequence features from said first set of organisms and depleted of sequence features from said second set of organism;
e. identifying at least one gene highly expressed in said second set of organisms that is not highly expressed in said first set of organisms and introducing into an open reading frame of said nucleic acid molecule at least a portion of said at least one gene highly expressed in said second set of organisms; and
f. optimizing intergenic sequence in said nucleic acid molecule by enriching said intergenic sequence with uptake signal sequences (USS) from said first set of organisms and depleting said intergenic sequence of USS from said second set of organisms;
and outputting an artificial sequence of said engineered nucleic acid molecule, thereby engineering a nucleic acid molecule.

2. The computerized method of claim 1, wherein said CUB is calculated by a tRNA adaptation index (tAI), by a codon adaptation index (CAI) or by typical decoding rate (TDR).

3. The computerized method of claim 1, wherein all codons of said nucleotide sequence that can be, are replaced with a synonymous codon selected for in said first set of organisms based on said CUB and deselected for in said second set of organisms based on said CUB.

4. The computerized method of claim 1, wherein said regulatory elements are promoters.

5. The computerized method of claim 1, wherein said highly expressed genes are selected based on a predetermined threshold of a percentage of all genes or are inferred based on CUB rankings of coding sequences of all genes in each organism.

6. (canceled)

7. The computerized method of claim 1, wherein selecting sequence motifs comprises employing a hidden Markov model.

8. The computerized method of claim 1, wherein engineering an artificial regulatory element comprises selecting an endogenous regulatory element from said first list which is highly enriched for said selected sequence motifs.

9. The computerized method of claim 8, wherein selecting an endogenous regulatory element comprises ranking said regulatory elements from said first list based on their enrichment with said selected sequence motifs and the significance of enrichment of said selected sequence motifs in said first list.

10. The computerized method of claim 9, wherein said ranking comprises using a k−1 order Markov model.

11. The computerized method of claim 8, further comprising producing at least one mutation in said endogenous regulatory element that produces at least one selected sequence motif.

12. The computerized method of claim 1, wherein said altering a sequence occurs within said coding region, or within a regulatory region that is required for or enhances expression of said coding region.

13. The computerized method of claim 12, wherein said altering is within said coding region and does not alter an amino acid sequence encoded by said coding sequence.

14. (canceled)

15. The computerized method of claim 1, wherein said DNA cleaving agent is selected from a restriction enzyme and a genome editing protein.

16. The computerized method of claim 15, wherein said genome editing protein is a clustered regulatory interspaced short palindromic repeats (CRISPR) protein.

17. The computerized method of claim 16, wherein said altering a sequence comprises producing a PAM sequence of a CRISPR protein and a spacer sequence expressed only by said second set of organisms.

18. The computerized method of claim 15, wherein said DNA cleaving agent is a restriction enzyme and said altering a sequence comprises producing at least one palindromic target sequences of a restriction enzyme expressed only by said second set of organisms or mutating a palindromic target sequence of a restriction enzyme expressed only by said first set of organisms.

19. The computerized method of claim 1, wherein generating an artificial ORI comprises performing hierarchical clustering of said extracted sequence features that promote replication from ORI from said first list of organisms and if a distance between clusters is greater than a predetermined threshold including all clusters in said nucleic acid molecule and if said distance is less than said predetermined threshold generating a single cluster related to all ORI sequences in all said clusters.

20. The computerized method of claim 19, comprising at least one of:
a. producing at least one mutation in said artificial ORI that produces a sequence feature from said first set of organisms or that removes a sequence feature from said second set of organisms; and
b. selecting at least one feature from at least one clusters from said first set of organisms and removing at least one feature from at least one cluster from said second set of organisms.

21. (canceled)

22. The computerized method of claim 1, wherein at least one of:
a. said at least one gene highly expressed in said second set of organisms is an essential gene;
b. said portion of said at least one gene highly expressed is said second set of organisms acts as an siRNA against said at least one highly expressed gene;
c. said nucleic acid molecule is a DNA molecule;
d. said nucleic acid molecule is a plasmid; and
e. said first set of organisms, said second set of organisms or both are bacteria.

23. (canceled)

24. (canceled)

25. (canceled)

26. (canceled)

27. (canceled)

28. An engineered nucleic acid molecule produced by a computerized method of claim 1.
